(19) United States
(12) Patent Application Publication
(10) Pub. No.: US 2015/0283311 A1
(43) Pub. Date: Oct. 8, 2015

(54) METHOD, APPARATUS, AND SYSTEM FOR EXPRESSION OF HUMAN BREAST MILK

(71) Applicant: Naia Health, Inc., Redwood City, CA (US)

(72) Inventors: Jeffery B. Alvarez, Redwood City, CA (US); Janica B. Alvarez, Redwood City, CA (US); Alex Goldenberg, San Francisco, CA (US); Greg Stahler, Belmont, CA (US)

(73) Assignee: Naia Health, Inc., Redwood City, CA (US)

(21) Appl. No.: 14/616,557

(22) Filed: Feb. 6, 2015

Related U.S. Application Data
(60) Provisional application No. 61/937,027, filed on Feb. 7, 2014.

(51) Int. Cl. A61M 1/06; H04L 29/08; G06F 19/00

(52) U.S. Cl. CPC A61M 1/06; G06F 19/3406; H04L 67/12; H04L 67/1095; A61M 2205/3584; A61M 2205/3327; A61M 2205/3306; A61M 2205/3379

(57) ABSTRACT

Systems, methods, and devices for milk expression are provided. In one aspect, a system includes an expression apparatus having an interface configured to engage a breast and an actuation assembly operably coupled to the interface. Actuation of the actuation assembly causes the interface to apply vacuum pressure against the breast to express milk from the breast. The system also includes a computing device configured to communicate with the expression apparatus via a data connection.

### CROSS-REFERENCE TO RELATED APPLICATIONS

The present application is a non-provisional of, and claims the benefit of U.S. Provisional Patent Application No. 61/937,027, filed on Feb. 7, 2014 [attorney docket no. 44936-704.101], the entire contents of which are incorporated herein by reference.

The subject matter of the present application is related to U.S. patent application Ser. No. 14/221,113, filed on Mar. 20, 2014, [attorney docket no. 44936-703.201], U.S. Provisional Patent Application No. 62/021,601, filed on Jul. 7, 2014 [attorney docket no. 44936-705.101], U.S. Provisional Patent Application No. 62/021,597, filed on Jul. 7, 2014 [attorney docket no. 44936-706.101], U.S. Provisional Patent Application No. 62/028,219, filed on Jul. 23, 2014 [attorney docket no. 44936-708.101], and U.S. Provisional Patent Application No. 62/052,941, filed on Sep. 19, 2014 [attorney docket no. 44936-709.101], the entire contents of which are incorporated herein by reference.

### BACKGROUND OF THE INVENTION

1. Field of the Invention

The present invention generally relates to medical devices and methods, and more particularly relates to devices and methods for expression and collection of human breast milk.

Breast pumps are commonly used to collect breast milk in order to allow mothers to continue breastfeeding while apart from their children. Currently, there are two primary types of breast pumps: manually-actuated devices, which are small, but inefficient and tiring to use; and electrically-powered devices, which are efficient, but large and bulky. Therefore, it would be desirable to provide improved breast pumps that are small and highly efficient for expression and collection of breast milk. Additional features such as milk production quantification, milk characterization, and communication with mobile devices are further desirable for enhanced user convenience. At least some of these objectives will be satisfied by the devices and methods disclosed below.

2. Description of the Background Art

The following US patents are related to expression and collection of human breast milk: U.S. Pat. Nos. 6,673,036; 6,749,582; 6,840,918; 6,887,210; 7,875,000; 8,118,772; and 8,216,179.

### SUMMARY OF THE INVENTION

The present invention generally relates to medical devices and methods, and more particularly relates to devices and methods for expression and collection of human breast milk.

In a first aspect of the present invention, a system for expression of milk from a breast is provided. The system may comprise an expression apparatus having an interface configured to engage a breast and an actuation assembly operably coupled to the interface. Actuation of the actuation assembly can cause the interface to apply vacuum pressure against the breast to express milk from the breast. The system further comprises a computing device configured to communicate with the expression apparatus via a data connection.

In many embodiments, the breast is a human breast. The interface can be configured to fluidly seal against the breast.

In many embodiments, the data connection utilizes wireless communication, near field communication, or a USB cable to transmit data between at a least a portion of the expression apparatus and the computing device. The computing device may be a smartphone, tablet, or personal computer.

In many embodiments, the expression apparatus further includes a sensing unit configured to generate measurement data indicative of one or more characteristics of milk expression. The measurement data can be transmitted to the computing device via the data connection. The computing device can include an application configured to analyze the measurement data. The computing device can transmit the measurement data to a server. The expression apparatus can further include a processing unit configured to analyze the measurement data to generate an analysis result, and a display unit configured to display the analysis result to a use. The analysis result can be displayed in a graph, chart, or table. The analysis result can be transmitted to a computing device via the data connection, and the computing device can display the analysis result to the user.

In many embodiments, the computing device can control at least one functionality of the expression apparatus via the data connection. The functionality can comprise one or more of power of the expression apparatus, vacuum pressure applied by the expression apparatus, or cycles per minute of the expression apparatus.

In many embodiments, a notification reminding a user to express milk may be transmitted to the computing device via the data connection. The notification can be transmitted to at least a portion of the expression apparatus via the data connection. Firmware updates can be transmitted to at least a portion of the expression apparatus via the data connection.

In many embodiments, the computing device can comprise a communication module in communication with a server via a network. A notification reminding a user to express milk may be transmitted from the server to the computing device. The computing device may comprise a cellular phone associated with a cellular phone number, and the notification may be transmitted by short message service (SMS) via the network to the cellular phone number.

In another aspect of the present invention, a method for measuring expression of milk from a breast is provided. The method comprises providing a breast milk expression apparatus having an interface, an actuation assembly operably coupled to the interface, and a sensing unit. The method further comprises engaging the interface with a breast, and actuating the actuation assembly, thereby causing the interface to apply vacuum pressure against the breast. The method further comprises expressing milk from the breast. The method may further comprise measuring a characteristic of milk expression using the sensing unit, in order to generate measurement data. The measurement data may be transmitted from the sensing unit to a computing device via a data connection.

In many embodiments, the measurement data may be stored in one or more data stores of the computing device. The measurement data can be analyzed via an application of the computing device to generate an analysis result, and the analysis result can be displayed to a user via the computing device. The analysis result can be displayed in a graph, chart, table, or any other visual, audible, or tactile indicator.

In another aspect of the present invention, a method for controlling expression of milk from a breast is provided. The method comprises providing a breast milk expression apparatus having an interface and an actuation assembly operably coupled to the interface. The method further comprises engaging the interface with a breast. A control signal may be received from a computing device via a data connection. The method may further comprise actuating the actuation assembly based on the control signal, causing the interface to apply vacuum pressure against the breast. The method further comprises expressing milk from the breast.

In another aspect of the present invention, an apparatus for expression of milk from a breast is provided. The apparatus comprises an interface configured to engage a breast and an actuation assembly operably coupled to the interface. Actuation of the actuation assembly can cause the interface to apply vacuum pressure against the breast to express milk from the breast. The apparatus can also include a communication module in communication with a server via a network.

In many embodiments, the network includes an Internet network. The apparatus can further include a sensing unit configured to generate measurement data indicative of one or more characteristics of milk expression, and the measurement data can be transmitted to the server via the network. The server can include an application configured to analyze the measurement data.

In many embodiments, the apparatus further includes a processing unit configured to analyze the measurement data to generate an analysis result, and a display unit configured to display the analysis result to a user. The analysis result can be transmitted to the server via the network. The analysis result can be displayed on a computing device in communication with the server.

In many embodiments, at least one functionality of the actuation assembly is controlled by an application on the server via the network. The functionality can include power of the actuation assembly, vacuum pressure applied by the actuation assembly, or cycles per minute of the actuation assembly.

In many embodiments, a notification reminding a user to express milk may be transmitted via the network to an email address. The notification reminding the user to express milk can be transmitted by short message service (SMS) via the network to a cellular phone number, such as by SMS from the server to a smartphone associated with the cellular phone number. The notification reminding the user to express milk can be transmitted to the communication module via the network. Firmware updates can be transmitted to the communication module via the network.

In another aspect, the present invention provides a method for measuring expression of milk from a breast. The method comprises providing a breast milk expression apparatus including an interface, an actuation assembly operably coupled to the interface, and a sensing unit. The interface may be engaged with a breast. The actuation assembly can be actuated, causing the interface to apply vacuum pressure against the breast. Milk is expressed from the breast. The sensing unit may be used to measure a characteristic of milk expression to generate measurement data. The measurement data may be transmitted to a server via a network.

In many embodiments, the server is a distributed computing server. The characteristic of milk expression can be measured by the sensing unit as the milk moves from the interface to a collection reservoir in fluid communication with the interface. The measurement data can be stored in one or more data stores associated with the server. The measurement data can be analyzed via an application on the server to generate an analysis result. The analysis result can be transmitted from the server to a computing device and displayed to a user via the computing device.

In another aspect of the present invention, a method for remotely controlling expression of milk from a breast is provided. The method comprises providing a breast milk expression apparatus comprising an interface and an actuation assembly operably coupled to the interface. The interface may be engaged with a breast. A control signal can be received from a server via a network. The actuation assembly may be actuated based on the control signal, causing the interface to apply vacuum pressure against the breast. Milk may be expressed from the breast.

In another aspect of the present invention, an apparatus for measuring expression of fluid from a breast is provided. The apparatus includes an interface configured to engage a breast and an actuation assembly operably coupled to the interface. Actuation of the actuation assembly causes the interface to apply vacuum pressure against the breast to express milk from the breast. The apparatus includes a sensing unit configured to generate measurement data indicative of volume of expressed fluid from the breast.

In many embodiments, the breast is a human breast. The interface can be configured to fluidly seal against the breast. The fluid can be breast milk or colostrum. The measurement data can be indicative of the volume per unit time, volume per stroke of the pump, or volume per pump power cycle of the expressed fluid.

In many embodiments, the interface includes a valve permitting the passage of the expressed fluid and the sensing unit comprises an accelerometer measuring motion of the valve. The measurement data can be generated based on the motion of the valve.

In many embodiments, the apparatus can further include a second interface configured to engage a second breast. Actuation of the actuation assembly may cause vacuum pressure to be applied alternatingly against the breast and the second breast to alternatingly express fluid from the breasts. The second interface can include a second valve permitting the passage of expressed fluid from the second breast and the sensing unit can include a second accelerometer measuring position of the second valve. The sensing unit can determine user motion based on motion detected by both the accelerometer and the second accelerometer. The user motion can be subtracted from motion detected by at least one of the accelerometer or the second accelerometer when determining position of at least one of the first or second valves.

In many embodiments, the interface may comprise an interface housing and a valve permitting passage of the expressed fluid. The sensing unit may comprise a first accelerometer coupled to the interface housing, and a second accelerometer coupled to the valve. The first accelerometer may be configured to measure a position of the interface housing. The second accelerometer may be configured to measure a position of the valve. The measurement data may be generated based on a position of the interface housing and a position of the valve. The sensing unit can determine background motion, based on motion detected by the first accelerometer. The background motion may be subtracted from motion detected by the second accelerometer when determine a position of the valve.

In many embodiments, the interface may be coupled to a reservoir configured to collect the expressed fluid. The sensing unit may be coupled to the reservoir. The reservoir may comprise a processing unit in communication with the sensing unit, and the processing unit may be configured to receive the measurement data generated by the sensing unit. The processing unit may further comprise a communication module configured to transmit the measurement data to a computing device via a data connection. The communication module of the processing unit may be configured to transmit the measurement data to a server via a network.

In many embodiments, the sensing unit includes a beam-break sensor configured to detect passage of the expressed fluid near one or more sensor components of the beam-break sensor. The measurement data can be generated based on a length of time the expressed fluid passes between the sensor components.

In many embodiments, the interface includes a valve permitting the passage of the expressed fluid and the sensing unit includes a charge-coupled device (CCD) configured to count drops of the expressed fluid passing through the valve. The measurement data can be generated based on one or more CCD images of the drops.

In many embodiments, the interface includes a tube permitting passage of the expressed fluid and the sensing unit includes a capacitive sensor configured to sense the expressed fluid contained in the tube. The interface can be coupled to a reservoir configured to collect the expressed fluid and the sensing unit can include a capacitive sensor configured to measure volume of the expressed fluid contained in the reservoir.

In many embodiments, the sensing unit includes a strain gauge configured to measure the volume of the expressed fluid. The interface can include a valve permitting the passage of the expressed fluid, and the strain gauge can be coupled to the valve and configured to determine displacement of the valve over time. The interface can be coupled to a reservoir configured to collect the expressed fluid, and the strain gauge can be coupled to the reservoir and configured to measure volume of the expressed fluid contained in the reservoir. The reservoir may comprise a bottom interior surface having a bellows element. The bellows element can be configured to minimize absorption by the bottom interior surface of a load placed on the bottom interior surface by the expressed fluid.

In many embodiments, the sensing unit includes a camera coupled to the interface and configured to capture one or more images of the expressed fluid. The apparatus can further include a processing unit configured to analyze the one or more images to determine the volume of the expressed fluid or other characteristics of the expressed fluid. The one or more images can be transmitted to a computing device configured to analyze the one or more images to determine the volume of the expressed fluid. The computing device can be a smartphone. The camera can be situated on a mobile device.

In many embodiments, the apparatus further comprises a processing unit and a control unit operably coupled to the actuation assembly to control at least one functionality of the actuation assembly. At least a subset of the measurement data may be transmitted as feedback to at least one of the processing unit and the control unit. The actuation assembly can include a pump, and the feedback can be used to adjust a vacuum stroke of the pump or cycles per minute of the pump to maintain optimal fluid expression.

In another aspect of the present invention, a method for measuring the volume of fluid expressed from a breast is provided. The method includes providing a breast fluid expression apparatus including an interface, an actuation assembly operably coupled to the interface, and a sensing unit. The interface is engaged with a breast. The actuation assembly is actuated, causing the interface to apply vacuum pressure against the breast. Fluid is expressed from the breast. Measurement data indicative of volume of the expressed fluid is generated via the sensing unit.

In many embodiments, the method further comprises changing an actuation parameter of the actuation assembly based on at least a subset of the measurement data. The actuation assembly is actuated based on the changed actuation parameter.

Other objects and features of the present invention will become apparent by a review of the specification, claims, and appended figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a perspective view of a pumping device, in accordance with embodiments;

FIG. 2 is a perspective view of a hydraulic pumping device, in accordance with embodiments;

FIG. 3 is a cross-section of a hydraulic pumping device, in accordance with embodiments;

FIG. 4 illustrates an actuation assembly coupled to a driving mechanism, in accordance with embodiments;

FIG. 5A-5B illustrate an actuation assembly coupled to a controller, in accordance with embodiments;

FIG. 6 is a cross-sectional view of a breast interface, in accordance with embodiments;

FIG. 7 is a cross-sectional view of another a breast interface, in accordance with embodiments;

FIG. 8A is a cross-sectional view of an integrated valve within a flexible membrane in an open position, in accordance with embodiments;

FIG. 8B is a cross-sectional view of an integrated valve within a flexible membrane in a closed position, in accordance with embodiments;

FIG. 9 is a cross-sectional view of a breast interface with a mechanical deformable member, in accordance with embodiments;

FIG. 10 is a cross-sectional view of a mechanical driver for a mechanical deformable member, in accordance with embodiments;

FIGS. 11A-11Q illustrate exemplary embodiments of sensors for detecting fluid;

FIG. 12 illustrates a controller and a mobile device, in accordance with embodiments;

FIG. 13 illustrates short range communication between a controller and a mobile device, in accordance with embodiments;

FIG. 14 is a schematic illustration of a pumping device in communication with a computing device and a server, in accordance with embodiments;

FIG. 15 is a graph illustrating the pump performance of an exemplary pumping device compared to a commercial device, in accordance with embodiments; and

FIG. 16 is a graph illustrating the pumping efficiency of an exemplary pumping device compared to a commercial device, in accordance with embodiments.

FIG. 17 illustrates a schematic diagram of a system for expression of milk;

FIG. 18 illustrates another exemplary embodiment of a system for expression of milk;

FIGS. 19A-19C illustrate exemplary displays on a computing device;

FIGS. 20A-20B illustrate exemplary displays in a milk expression system;

FIG. 21 illustrates the use of a feedback control loop to control a milk expression device; and

FIG. 22 illustrates a dual expression system.

### DETAILED DESCRIPTION OF THE INVENTION

Specific embodiments of the disclosed systems, devices, and methods will now be described with reference to the drawings. Nothing in this detailed description is intended to imply that any particular component, feature, or step is essential to the invention. Although the present invention primarily relates to breast milk, any description herein of expression and collection of breast milk can also be applied to other types of fluids expressed from the breast, such as colostrum. Furthermore, the disclosed embodiments may be used in other applications, particularly applications involving the creation and transmission of a pressure differential, such as in the treatment of sleep apnea and/or other remote pressure needs.

The systems, devices, and methods of the present invention provide improved pumping devices for the expression and collection of breast milk, such as human breast milk. Contrary to existing devices, the mechanisms described herein enable the development of smaller and more efficient electrical pumping devices, thereby enhancing convenience and ease of use. Additionally, at least some of the exemplary embodiments disclosed herein incorporate sensors for measuring characteristics of milk expression. The resultant data can be used, for instance, as feedback for improving pumping efficiency, as well as to provide information and/or analytics relevant to milk expression to the user. Furthermore, in preferred embodiments, the data can be transmitted to another device in communication with the pumping device, thereby enabling control, display, and/or analysis of milk expression to be performed remotely.

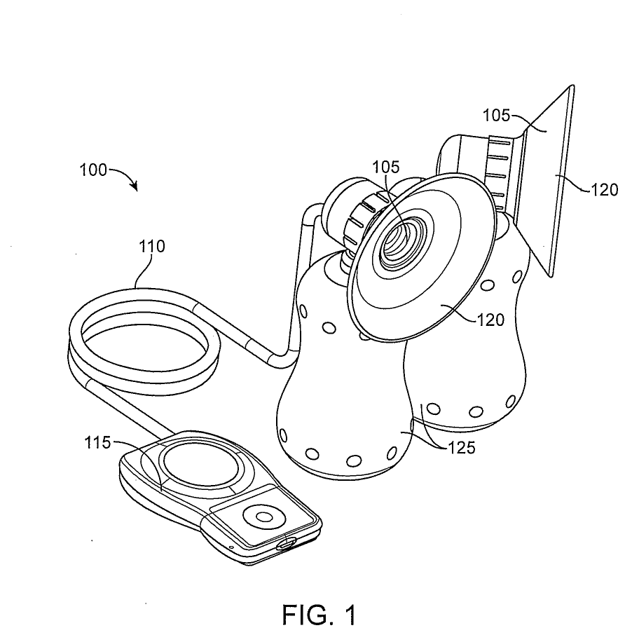

FIG. 1 illustrates an exemplary embodiment of the present invention. Pumping device 100 (also known as an “expression apparatus”) includes breast interfaces 105, a tube 110, and a controller 115 (sometimes also referred to as a “pendant unit”) operatively coupled to breast interfaces 105 through tube 110. Breast interfaces 105 include resilient and conformable flanges 120, for engaging and creating a fluid seal against the breasts, and collection vessels 125. Controller 115 houses the power source and drive mechanism for pumping device 100, and also contains hardware for various functions, such as controlling pumping device 100, milk production quantification, and communication with other devices, as described in further detail herein. Tube 110 transmits suitable energy inputs, such as mechanical energy inputs, from controller 115 over a long distance to breast interfaces 105. Breast interfaces 105 convert the energy inputs into vacuum pressure against the breasts in a highly efficient manner, resulting in the expression of milk into collection vessels 125. The device 100 may further comprise one or more sensors configured to track various characteristics of the collected fluid, as described in further detail herein. Power may be provided to the one or more sensors via a connection to the controller 115, or to another source of power. In embodiments in which the one or more sensors are coupled to one or more portions of the breast interfaces 105 or collection vessels 125, the sensors may be further coupled to the controller 115 via one or more communication lines configured to transmit signals between the sensors and the controller.

Hydraulic Pumping Device

Hydraulic systems can reduce pumping force requirements, and therefore also reduce the size of the pumping device, while maintaining high pumping efficiencies. In a preferred embodiment, the pumping device can utilize a hydraulic system to generate a pressure differential against the breast for the expression and collection of milk.

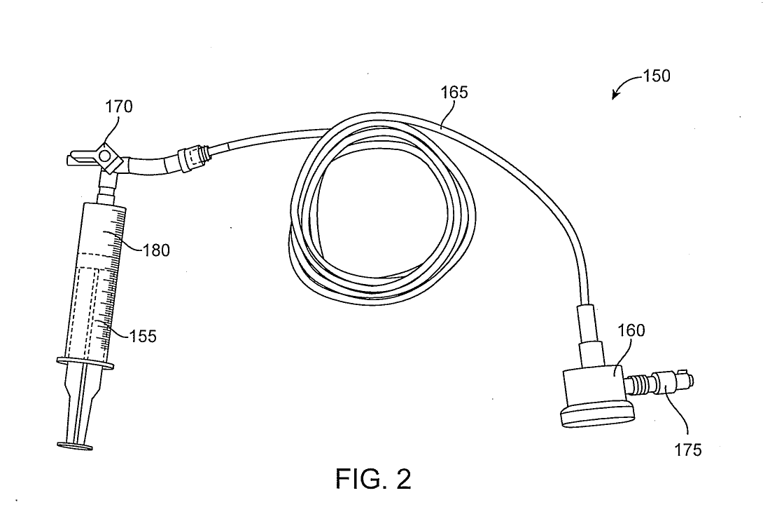

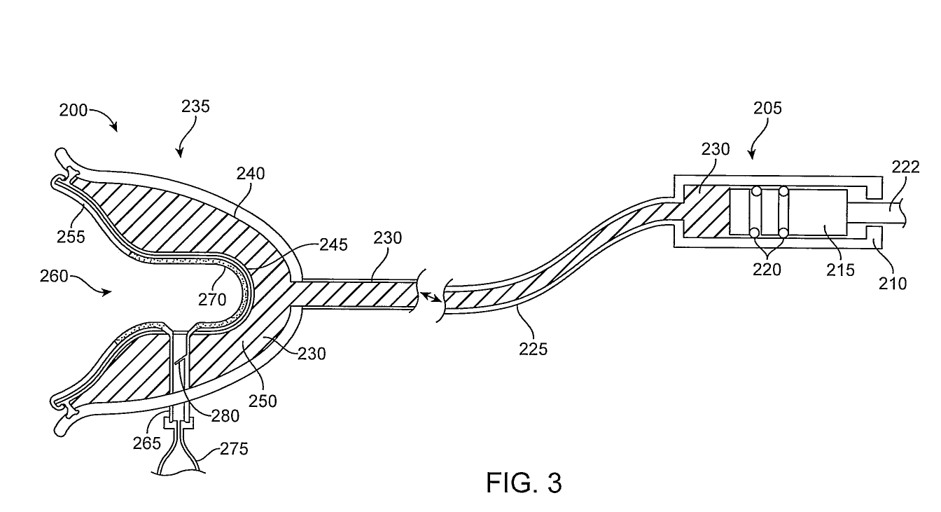

Exemplary hydraulic pumping devices are depicted in FIGS. 2 and 3. FIG. 2 illustrates a pumping device 150 with a syringe 155 fluidly coupled to breast interface 160 by tube 165. Syringe 155 is coupled to tube 165 through a three-way valve 170. Breast interface 160 contains an exit port 175. The syringe 155 drives a fluid 180 contained within tube 165 against a flexible member contained within breast interface 160 to create the pressure differential necessary for milk expression from the breast.

FIG. 3 illustrates another embodiment of a pumping device 200. The actuation assembly 205 includes an assembly housing 210, a driving element 215, seals 220, and a shaft 222. Driving element 215 is operatively coupled to a controller, such as controller 115, through shaft 222. The tube 225 contains a fluid 230 and is fluidly coupled to the actuation assembly 205 and the breast interface 235. The breast interface 235 consists of an interface housing 240, a flexible membrane 245, a reservoir 250, a sealing element 255, an expression area 260, and a drain port 265. The sealing element 255 includes deformable portion 270. Alternatively, the flexible membrane 245 may comprise a sealing element 255 having a deformable portion 270, the flexible membrane 245 configured to function as a sealing element by fluidly sealing against the breast engaged into the breast interface 235. The drain port 265 is coupled to a collection vessel 275 and includes a flap valve 280.

Actuation assembly 205 displaces fluid 230 contained within tube 225, which can be a flexible line. Fluid 230 occupies reservoir 250 within breast interface 235 and is coupled with flexible membrane 245. Preferably, the couplings between the flexible membrane 245, sealing element 255, and interface housing 240 are fluid-tight couplings, such that the fluid 230 is contained within the reservoir 250 and cannot infiltrate into the expression area 260. Flexible membrane 245 transmits vacuum pressure from fluid 230 to the deformable portion 270 of sealing element 255. When a breast is engaged into and fluidly sealed with breast interface 235 by sealing element 255, displacement of the actuation element 215 produces substantial vacuum pressure against the breast through flexible membrane 245 and deformable portion 270, resulting in the expression of breast milk into expression area 260. Alternatively, the flexible membrane 245 may comprise the sealing element 255 having a deformable portion 270, such that the flexible membrane 245 forms a fluid seal against the breast engaged into the breast interface 235, and transmits vacuum pressure from fluid 230 to a deformable portion of the flexible membrane 245. The expressed milk drains through drain port 265 into collection vessel 275. Drain port 265 is configured with a flap valve 280 to provide passage of milk while maintaining vacuum pressure in expression area 260. Collection vessel 275 can be any suitable container, such as a bottle or a bag. In many embodiments, collection vessel 275 is removably coupled to flexible membrane 245. Collection vessel 275 can be coupled directly or remotely via any suitable device such as extension tubing. Preferably, the collection vessel can be quickly decoupled from the other components of the pumping device 200 (e.g., for milk storage, cleaning, etc.).

The fluid for the hydraulic pumping device can be any suitable fluid, such as an incompressible fluid. In many embodiments, the incompressible fluid can be a liquid, such as water or oil. In many embodiments, the fluid can be a fluid having properties such that the vacuum pressure exerted on the fluid by the pumping device does not result in outgassing of the fluid. Alternatively, the fluid can be any suitable gas, such as air. Any liquid or gas suitable for use with hydraulic systems can be used for the hydraulic pumping devices described herein.

Actuation Mechanism

Many actuation mechanisms known to those of skill in the art can be utilized for the actuation assembly 205. Actuation assembly 205 can be a piston assembly, a pump such as a diaphragm pump, or any other suitable actuation mechanism. The optimal configuration for actuation assembly 205 can depend on a number of factors, such as: vacuum requirements; size, power, and other needs of the pumping device 200; and the properties of the fluid 230, such as viscosity, biocompatibility, and fluid life requirements.

FIG. 3 illustrates an exemplary embodiment in which actuation assembly 205 is a piston assembly and driving element 215 is a piston. Actuation assembly 205 includes seals 220, such as O-rings, rolling diaphragm seals, or wiper seals, sealing against assembly housing 210 to prevent undesired egress of fluid 230 and to enable driving of fluid 230.

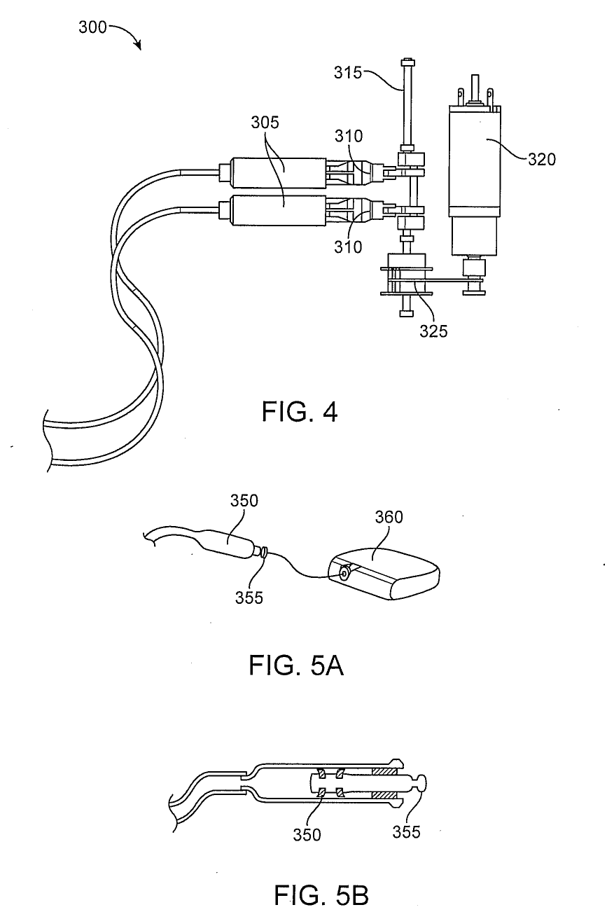

FIG. 4 illustrates another exemplary embodiment of an actuation assembly 300 including a pair of pistons 305.

In preferred embodiments, the actuation assembly includes a driving element powered by a suitable driving mechanism, such as a driving mechanism residing in controller 115. Many driving mechanisms are known to those of skill in the art. For instance, the driving element, such as driving element 215, may be actuated electromechanically by a motor, or manually by a suitable user-operated interface, such as a lever. Various drive modalities known to those of skill in the art can be used. In particular, implementation of the exemplary hydraulic pumping devices as described herein enables the use of suitable drive modalities such as direct drive and solenoids, owing to the reduced force requirements of hydraulic systems.

Referring now to the exemplary embodiment of FIG. 4, the pistons 305 include couplings 310 to a crankshaft 315. The crankshaft 315 is operatively coupled to a motor 320 through a belt drive 325. The crankshaft 315 drives the pair of pistons 305 with the same stroke timing in order to apply vacuum pressure against both breasts simultaneously, a feature desirable for increased milk production. Alternatively, the crankshaft 315 can drive the pair of pistons 305 with any suitable stroke timing, such as alternating or offset stroke cycles. Alternating or offset stroke cycles can have the benefit of reducing the power requirement of the motor 320.

The driving mechanism can be powered by any suitable power source, such as a local battery or an AC adaptor. The driving mechanism can be controlled by hardware, such as onboard electronics located within controller 115.

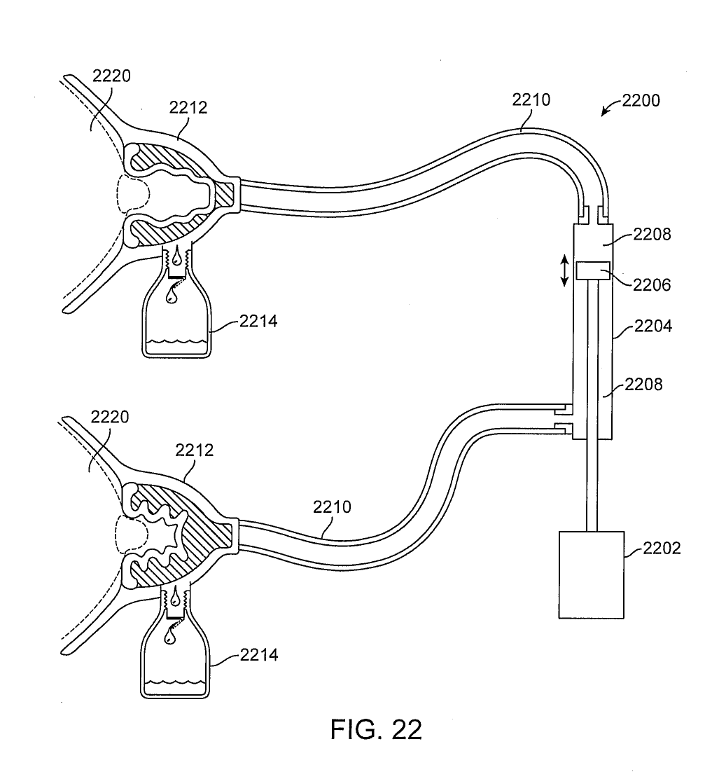

FIG. 22 illustrates another embodiment of an alternating pump system 2200. The system 2200 includes dual expression devices with an interface 2212 sized and shaped to conform to the target tissue, here a breast 2220. A reservoir 2214 is threadably or otherwise coupled to the expression device. A hydraulic line 2210 fluidly couples each expression device to a hydraulic piston assembly 2204 which has an incompressible fluid such as oil in a piston chamber and an actuatable piston 2206. One hydraulic line 2210 is coupled to the high pressure side 2208 of the hydraulic piston, and the other hydraulic line is coupled to the lower pressure side 2208 of the piston. A motor 2202 actuates the piston 2206. Thus, in operation, as the piston is actuated the high pressure side creates a higher pressure in one of the expression devices and a lower pressure in the other expression device. The lower pressure expression device results in a vacuum which causes milk expression, while the high pressure side does not express milk. Then, as the piston reaches the end of its stroke, and reciprocates in the opposite direction, the high and low pressure sides are reversed, thereby causing expression of milk on the opposite side and no expression on the original side. This process allows milk to be collected in an alternating fashion. The expression devices, reservoirs in this system may be any of the components disclosed elsewhere in this disclosure.

FIGS. 5A-5B illustrate an exemplary embodiment of an actuation assembly 350 that includes releasable coupling 355. FIG. 5A is an isometric view of the actuation assembly 350 and controller 360 coupled via a releasable coupling 355. FIG. 5B is a cross-sectional view of the actuation assembly 350 comprising a releasable coupling 355. Preferably, actuation assembly 350 is releasably coupled to a controller 360 and the driving mechanism housed therein. The coupling can be a mechanical coupling or any suitable quick release mechanism known to those of skill in the art. The releasably coupled design allows for flexibility in the configuration and use of the pumping device. For instance, user comfort can be improved through the use of differently sized breast interfaces for compatibility with various breast sizes. Additionally, this feature enables a common pumping device to be used with interchangeable breast interfaces, thus reducing the risk of spreading pathogens. Furthermore, the releasable coupling enables easy replacement of individual parts of the pumping device.

Flexible Membrane

In many embodiments, such as the embodiment depicted in FIG. 3, the flexible membrane 245 is located within breast interface 235 and disposed over at least portion thereof, forming reservoir 250 between the interface housing 240 and the flexible membrane 245. Preferably, the flexible membrane 245 deforms substantially when subject to the negative pressures created when the fluid 230 is displaced from reservoir 250 by actuation assembly 205. The amount of deformation of the flexible membrane 245 can be controlled by many factors, (e.g., wall thickness, durometer, surface area) and can be optimized based on the pumping device (e.g., pump power, vacuum requirements).

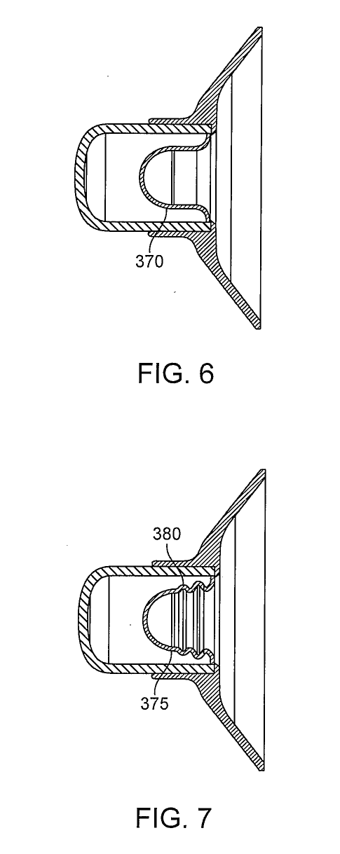

FIG. 6 illustrates an exemplary flexible membrane 370 with a specified thickness and durometer.

FIG. 7 illustrates another embodiment of flexible membrane 375 with corrugated features 380 for increased surface area.

Suitable materials for the flexible membrane are known to those of skill in the art. In many embodiments, the flexible membrane can be made of a material designed to expand and contract when subject to pressures from the coupling fluid such as silicone, polyether block amides such as PEBAX, and polychloroprenes such as neoprene. Alternatively, the flexible membrane can be fabricated from a substantially rigid material, such as stainless steel, nitinol, high durometer polymer, or high durometer elastomer. In these embodiments, the rigid material would be designed with stress and/or strain distribution elements to enable the substantial deformation of the flexible membrane without surpassing the yield point of the material.

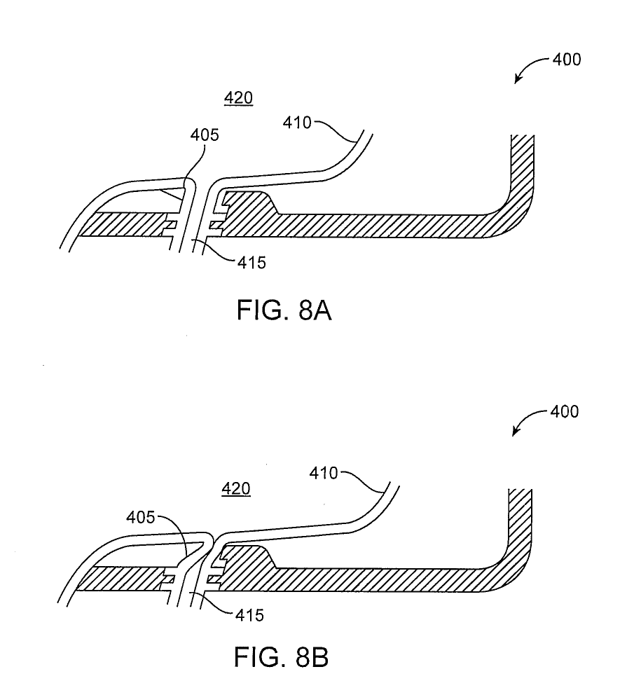

FIGS. 8A and 8B illustrate preferred embodiments of a breast interface 400 in which an exit valve 405 is integrated into the flexible membrane 410 to control the flow of expressed milk through exit port 415. The exit valve 405 is opened to allow fluid flow when the flexible membrane 410 is relaxed, as shown in FIG. 8A, and is closed to prevent fluid flow when the flexible membrane 410 is deformed, as shown in FIG. 8B. The exit valve 405 enables substantial vacuum pressure to be present in expression area 420 during extraction, while allowing milk to drain during the rest phase of the pump stroke. While many conventional breast pump valves function on pressure differentials alone, the exit valve 405 can preferably be configured to also function on the mechanical movement of flexible membrane 410. Incorporation of an integrated exit valve 405 with mechanical functionality as described herein can improve the sealing of the breast interface 400 during vacuum creation. Furthermore, the implementation of an exit valve integrally formed within the flexible membrane 410 such as exit valve 405 reduces the number of parts to be cleaned.

### Mechanical Pumping Device

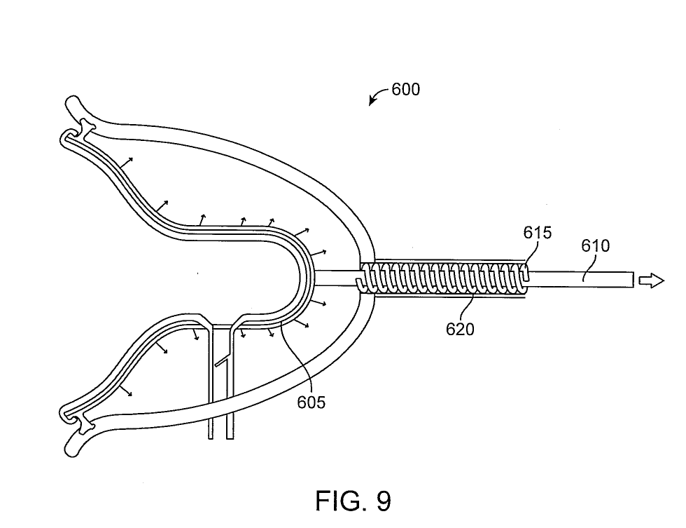

FIG. 9 illustrates an alternative embodiment of a breast interface 600 in which a mechanical deformable member 605 can be used in place of a flexible membrane. The mechanical deformable member 605 can be constructed from similar techniques as those used for the flexible membrane as described herein. The mechanical deformable member 605 is coupled to a tensile element 610. In some instances, tensile element 610 is disposed within an axial load absorbing member 615. The axial load absorbing member 615 is disposed within tube 620. Preferably, tensile element 610 is concentrically disposed within axial load absorbing member 615 and axial load absorbing member 615 is concentrically disposed within tube 620. Alternative arrangements of tensile element 610, axial load absorbing member 615, and tube 620 can also be used.

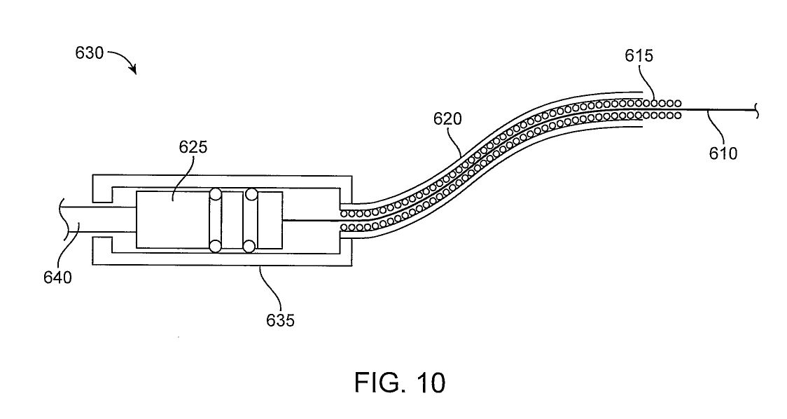

FIG. 10 illustrates the tensile element 610 coupled to driving element 625 of an actuation assembly 630 within an assembly housing 635. Driving element 625 is operatively coupled to a driving mechanism, such as a driving mechanism housed within a controller, through shaft 640. Axial load absorbing member 615 within tube 620 is fixedly coupled to the assembly housing 635. Displacement of the driving element 625 transmits tensile force through tensile element 610 to the mechanical deforming member 605 to create vacuum pressure against the breast. The driving element 625 can be actuated by a suitable driving mechanism, such as the embodiments previously described herein.

The tensile element 610 can be any suitable device, such as a wire, coil, tube, braid, rope, or any combination thereof. For example, with tensile element 610 can be a small nitinol wire with stainless steel braid disposed around it. The tensile element 610 can be made from any suitable material having high tensile strength, such as metals, polymers, or elastomers. Axial load absorbing member 615 can be made from any suitable axially stiff materials, such as metals or polymers, and can be configured into any suitable axially stiff geometry, such as a tube or coil.

### Fluid Collection and Quantification System

In many instances, it can be desirable to measure and track various characteristics of the collected fluid such as milk expression and collection, such as the amount of milk production (e.g., volume, weight), expression frequency (e.g., time, date), and/or expression duration. In existing approaches, the tracking of milk production is commonly accomplished by manual measurements and record-keeping. Exemplary embodiments of the devices described herein may provide digital-based means to automatically measure and track milk production for improved convenience, efficiency, and accuracy. For example, sensors can be used to measure the volume of expressed milk. In preferred embodiments, the volume can be measured as volume per unit time, volume per pump stroke (e.g., stroke of the actuation assembly), or volume per pump power cycle (e.g., power cycle of the actuation assembly).

In exemplary embodiments, the pumping devices described herein include one or more sensors for generating measurement data indicative of one or more characteristics of milk expression, such as the volume of expressed milk. Any description herein pertaining to measurement of volume can also be applied to measurements of other characteristics, and vice-versa. Any suitable type of sensor can be used, such as accelerometers, Hall effect sensors, photodiode/LED sensors, CCD sensors, cameras and other imaging devices, capacitive sensors, strain gauges, etc., and such sensors can be used in any number and combination. The sensors can be positioned at any location suitable for monitoring fluid flow from the breast, such as on or near a breast interface (e.g., the expression area 260, drain port 265, collection vessel 275). In embodiments where milk is concurrently expressed from a pair of breasts via a pair of breast interfaces, sensors can be located on or near both breast interfaces, or on or near only one of the breast interfaces. The sensors may be integrally formed with or permanently affixed to the pumping device. Alternatively, the sensors may be provided separately and coupled to the pumping device prior to use.

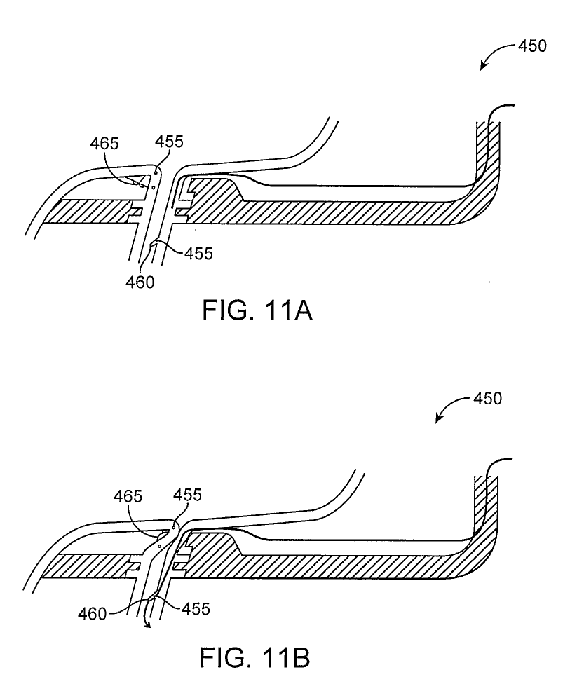

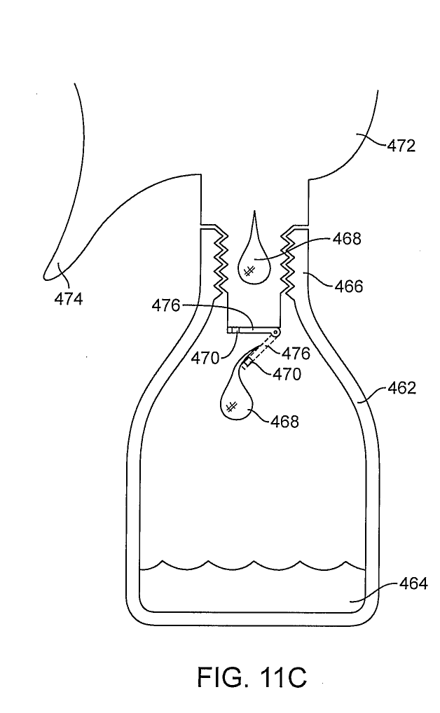

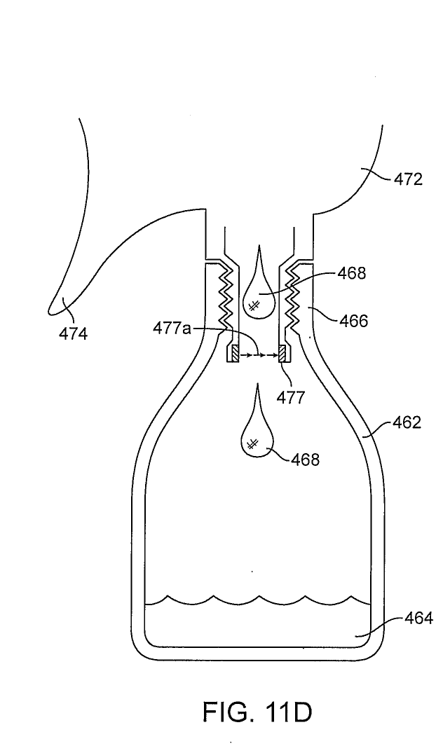

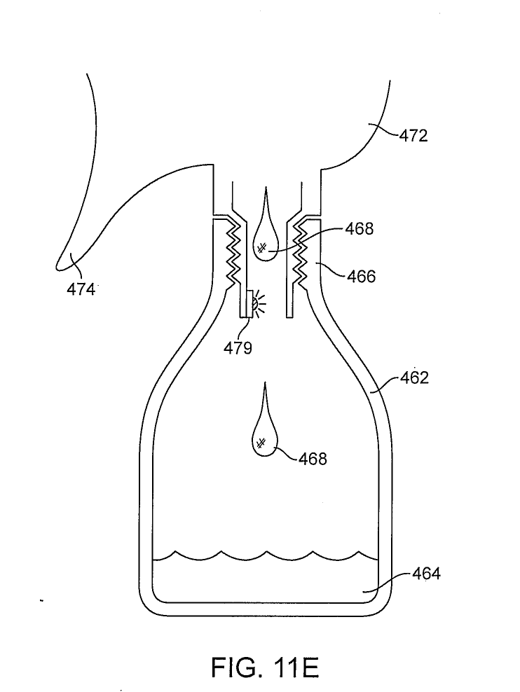

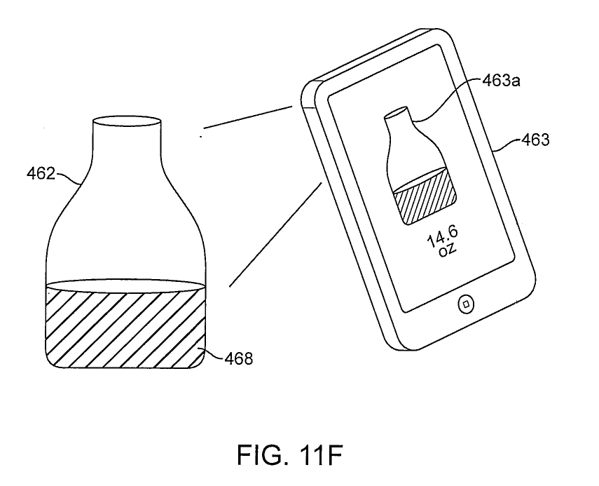

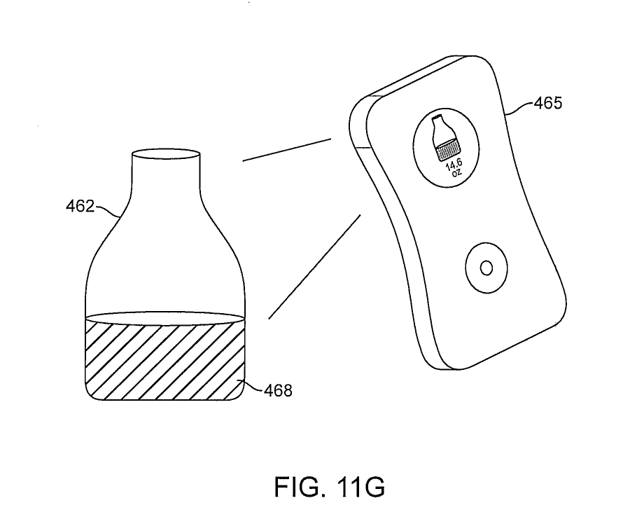

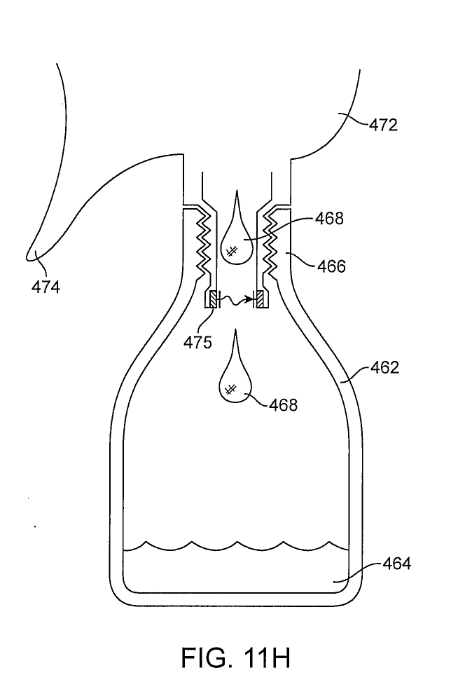

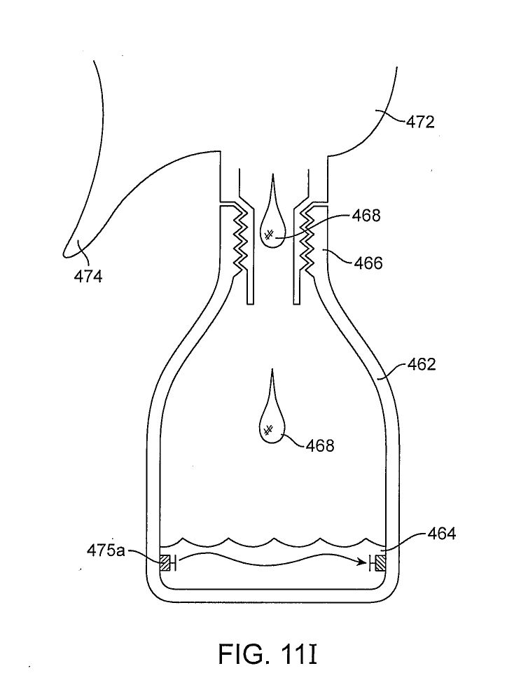

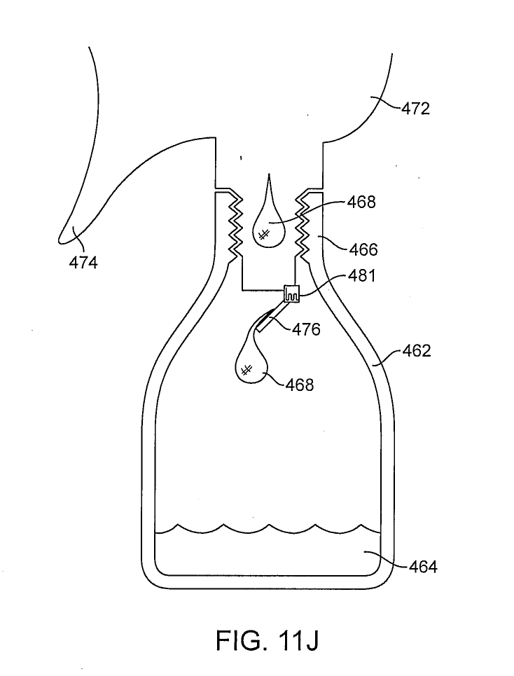

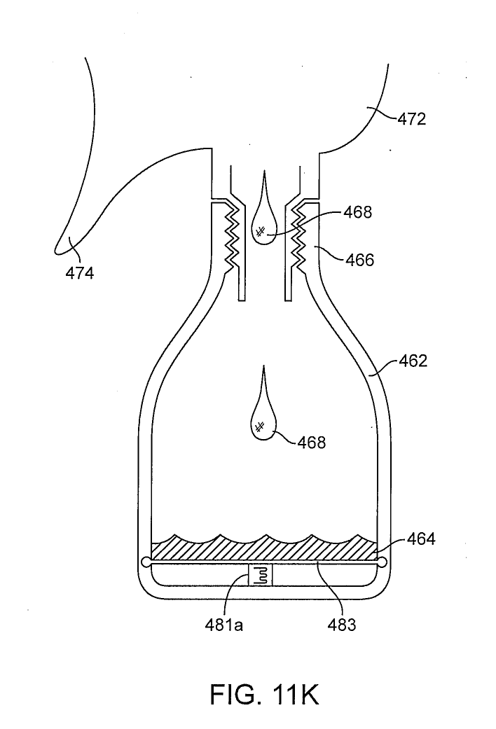

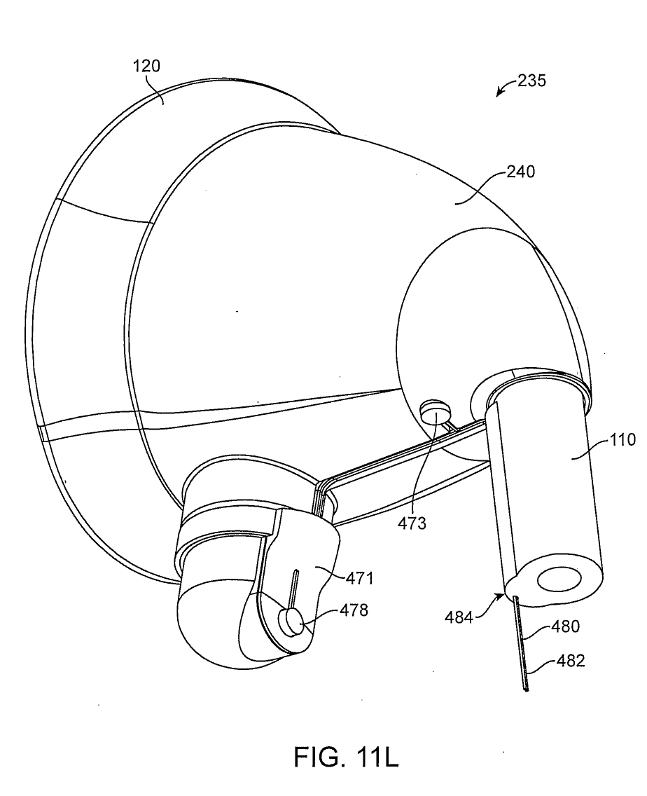

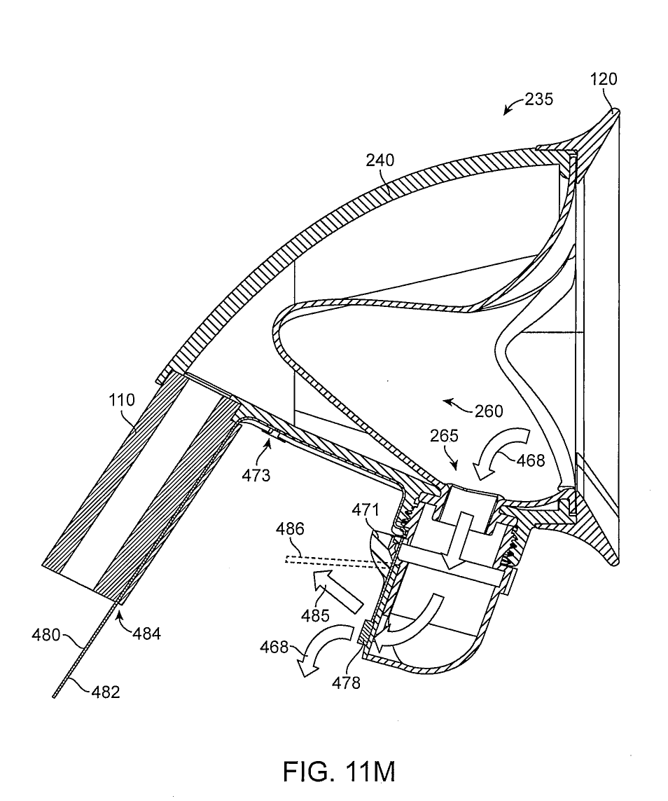

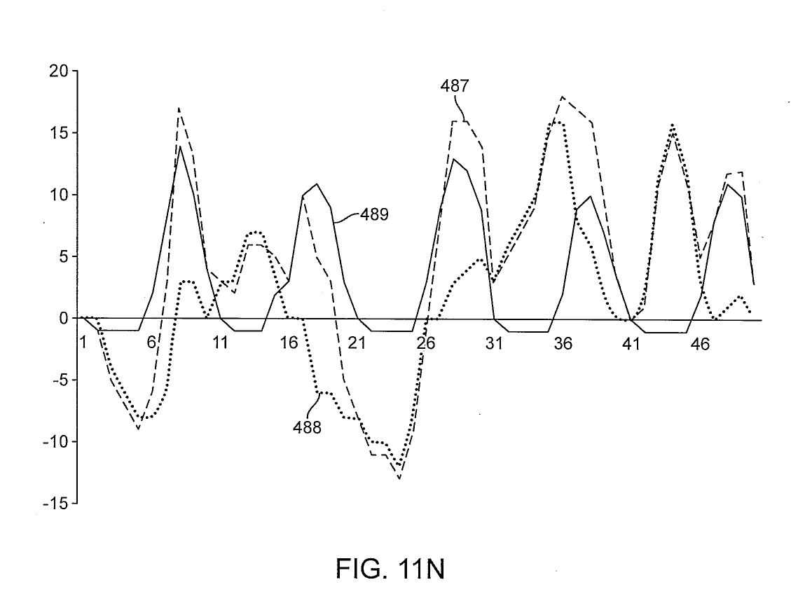

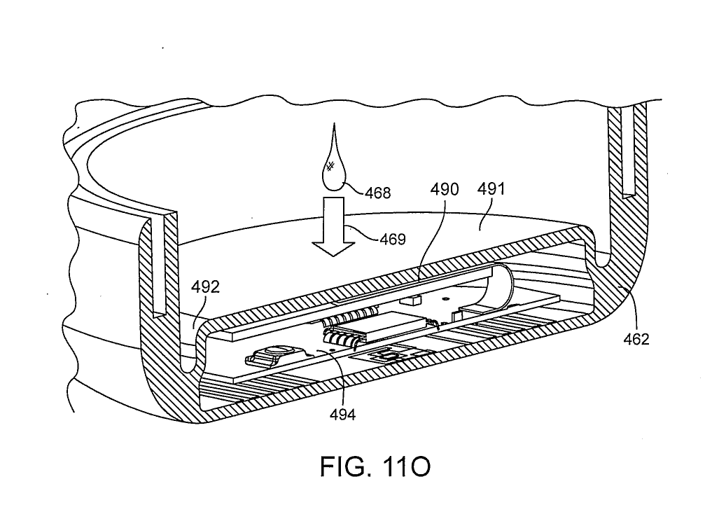

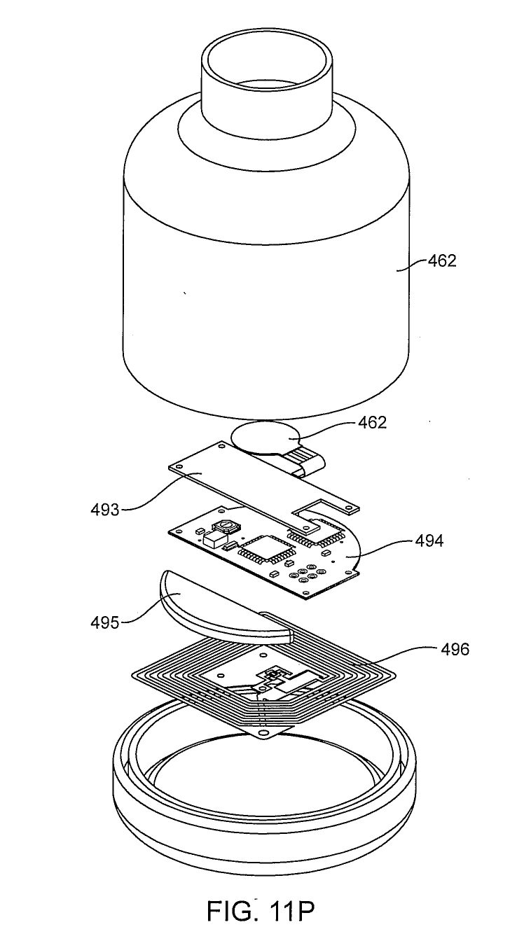

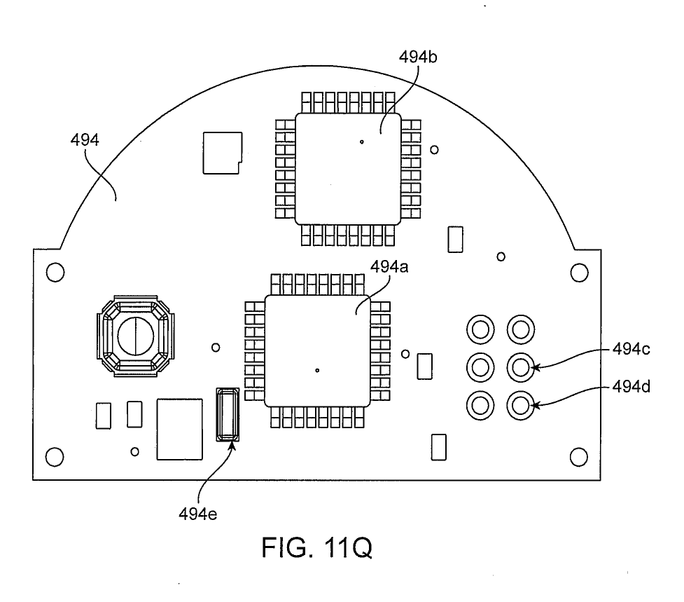

FIGS. 11A and 11B illustrate exemplary embodiments of a breast interface 450 with valve-integrated sensors 455. Sensors 455 are preferably located in a valve, such as the flap valve 460, but may also be located in exit valve 465, or any other valve (e.g., on or near the collection vessel) that is opened by fluid flow. In exemplary embodiments, the sensor 455 includes an accelerometer measuring the position and/or motion of the valve, such as a length of time that the valve is opened, and the resultant measurement data can be interrogated to quantify the fluid flow. Preferably, the breast interface 450 is used in conjunction with a second, identical breast interface to concurrently express milk from a pair of breasts (e.g., simultaneously, alternatingly, or sequentially). A pair of accelerometers can be used to detect the position and/or motion of the corresponding valve in each interface. In some instances, movements of the user may cause the accelerometers to produce motion signals that are erroneously interpreted as valve motion. Accordingly, in preferred embodiments, suitable approaches are used to distinguish between signals resulting from motion of the user and signals generated by motion of the valves. For example, the pumping device can be configured to alternatingly express milk from each breast, such that the corresponding valves are also opened alternatingly. Consequently, motion detected simultaneously from both accelerometers can be regarded as resulting from user motion, rather than from valve motion. The user motion can be subtracted from the total motion signal obtained by the accelerometers in order to obtain the valve motion, and thereby determine the position of each valve. Alternatively or in combination, the sensor 455 may comprise a set of background motion accelerometers in addition to the set of valve accelerometers, wherein the background motion accelerometers are configured to measure background motion including motion of a user, as described in further detail herein. The background motion measured by the background motion accelerometers may be subtracted from the motion measured by the valve accelerometers, in order to obtain the isolated valve motion.

FIG. 11C illustrates an embodiment with an accelerometer 470 more clearly. The accelerometer 470 is coupled to a valve 476 on the output of the expression device 472 which has a breast interface 474 (sometimes also referred to as a distal assembly in this specification). The valve 476 may be a flap valve, a duckbill valve, or the like. The expression device and breast interface may be any of the embodiments disclosed herein. As breast milk 468 is expressed, it collects at the output of the device. When enough fluid is collected, flap valve 470 opens, and the milk 468 drains into reservoir 462 and collects in a layer 464 therein. The reservoir 462 is preferably threadably connected to the expression device 472 so that it may easily be attached and detached. Movement of the valve 476 is tracked using accelerometer 470. Data from the accelerometer is then processed, transmitted or displayed using any of the methods or means disclosed herein.

FIGS. 11L-11N illustrate an exemplary embodiment having a background motion accelerometer 473 and a valve motion accelerometer 478. FIG. 11L is an isometric view of the exemplary embodiment. FIG. 11M is an side cross-sectional view of the exemplary embodiment. The background motion accelerometer 473 may be coupled to a portion of the breast interface 235, for example to the housing 240 of the breast interface. The valve motion accelerometer 478 may be coupled to a valve 471, wherein the valve 471 may be a flap valve, a duckbill valve, or any other valve configured to open in response to pressure or weight exerted by the expressed milk 468. The background motion accelerometer 473 and valve motion accelerometer 478 may be coupled to a power source, for example the controller 115, through a power line 482. The background motion accelerometer 473 and valve motion accelerometer 478 may be further coupled to a processing device or a communication module of a pump control unit, such as the controller 115, through a communication line 480. Alternatively or in combination, the accelerometers may be coupled to a communication module disposed on a portion of the breast interface or of a reservoir, wherein the communication module is configured to communicate wirelessly with the controller or a computing device. The power line 482 and communication line 480 may comprise one or more wires, and may be disposed within different channels or within the same channel 484 of the flexible tubing 110, coupling the breast interface 235 to an actuation assembly of the pumping device. The background motion accelerometer 473 may be disposed on a surface of the housing 240 so as to be positioned close to the power line 482 and communication line 480. Similarly, the valve motion accelerometer 478 may be disposed on a surface of the valve 471 so as to be positioned close to the power and communication lines. The valve 471 may be arranged in a configuration such that the valve accelerometer 478 disposed thereon can be positioned close to the power and communication lines. Preferably, the background motion accelerometer 473 may be disposed in a location and orientation that align its sensing axes to the axes of the valve accelerometer 471, so as to optimize the consistency of the positional data generated by the accelerometers.

As shown in FIG. 11M, the expressed milk 468 can enter the expression area 260 of the breast interface 235, and subsequently enter the drain port 265 coupled to a collection vessel. The fluid flow of the milk 468 can create pressure against the valve 471, causing the valve 471 to open. For example, the valve 471 may be displaced in the direction shown by arrow 485, to the configuration 486. The movement of the valve 471 may be tracked by the valve motion accelerometer 478. The valve motion accelerometer 478 may often measure background motion unrelated to the motion of the valve 471, such as user motion, in addition to the motion of the valve 471. In order to normalize the measurement of the valve motion accelerometer 478 against the background motion, the background motion accelerometer 473 can be configured to track the overall movement of the pumping device in space, so as to measure the background motion of the device unrelated to the motion of the valve 471. Data generated by the accelerometers 473 and 478 may be transmitted via the communication line 480 to a communication module of a pump control unit, wherein the communication module may be configured to transmit the data to a processing device, either of the pump control unit or of another computing device, for data analysis and/or display. Alternatively or in combination, the data generated by the accelerometers may be transmitted wirelessly, via a communication module integrated with a portion of the breast interface or of the reservoir.

FIG. 11N shows an exemplary graph of motion signals generated by the background motion accelerometer 473 and valve motion accelerometer 478. As shown in the graph, the valve signal 487 generated by the valve motion accelerometer 478 differs from the background signal 488 generated by the background motion accelerometer 473. In order to exclude or minimize the contribution of background motion to the measured valve motion, the background signal 488 may be subtracted from the valve signal 487 to generate a normalized valve signal 489.

In other exemplary embodiments, the pumping devices described herein can utilize one or more beam-break sensors (e.g., infrared-based, laser-based, etc.) situated at a suitable location in the pumping device (e.g., in or near a valve, an exit port, or other component permitting fluid passage). The beam-break sensor can include a plurality of sensor components and can be configured to detect passage of fluid between or near one or more of the components. Preferably, the sensor can be configured to generate a signal when the expressed fluid breaks a beam by passing between a beam emitter and a beam detector. The resultant signal can be used to produce measurement data indicative of the volume of expressed fluid. For example, the measurement data can be based on the length of time the fluid passes between or near the sensor components.

FIG. 11D illustrates an exemplary embodiment of a milk expression device that employs a beam break sensor 477. The expression device 472 includes a breast interface 474 and a reservoir 462. The reservoir is threadably or otherwise coupled 466 to the expression device. Any of the exemplary embodiments of expression devices, interfaces, reservoirs, etc. disclosed in this specification may be used in this exemplary system. A beam-break sensor 477 is disposed adjacent the output of the expression device, and thus as droplets 468 of milk drain from the expression device outlet into reservoir 462, they break the light beam 477a, allowing measurement of the fluid expressed. The fluid collects in a layer 464 in reservoir 462. The data from the sensor can then be processed, transmitted, or otherwise displayed using any of the methods disclosed herein.

In another exemplary embodiment, the pumping devices described herein can include one or more image sensors for capturing images of the fluid in order to quantify the expression volume, such as a charge-coupled device (CCD), active pixel sensors in complementary metal-oxide-semiconductor (CMOS), or a camera. The image sensors may be integrated with or coupled to a suitable portion of the pumping device. Conversely, the image sensors can be located on another device separate from the pumping device, such as a smartphone or other mobile device. In exemplary embodiments, the breast interface includes a valve permitting the passage of expressed fluid, as previously described herein, and a suitable image sensor is positioned on or near the valve in order to capture images of fluid passing through the valve. Preferably, the image sensor is operably coupled to a processing unit configured to analyze the image data (e.g., using a suitable image analysis algorithm) in order to determine the fluid volume. For example, the image sensors can be used to capture images of drops of fluid, and the images can be analyzed to count the number of drops. In some instances, the image data can be transmitted to a computing device (e.g., a smartphone) for analysis, as described in further detail below.

FIG. 11E illustrates an exemplary embodiment having a CCD or CMOS device 479 adjacent an outlet of the expression device. The expression device 472 includes interface 474 and reservoir 462, either of which may be any of the embodiments disclosed herein. As milk 468 is expressed, it passes through the outlet of the expression device past CCD or CMOS 479 which detects the fluid and allows quantification thereof as previously described. The milk 468 then accumulates in a layer 464 in reservoir 462. Data from the device 479 may then be processed, transmitted, or otherwise displayed using any of the methods disclosed herein.

FIG. 11F illustrates an exemplary embodiment that uses an image of the reservoir to characterized the expressed milk. Once milk 468 has been collected in reservoir 462, the reservoir may optionally be detached from the expression device. A mobile phone may then be used to take a photo 463a of the reservoir which has a suitable application for analyzing the photo and determining how much milk has been expressed, as well as optionally providing other details about the expressed milk. The data is processed, transmitted, or otherwise displayed using any of the methods disclosed herein.

FIG. 11G illustrates an alternative embodiment of a photo sensor system. After milk 468 has been expressed and collected in a reservoir 462, a camera in the pump control unit 465 may be used to obtain an image of the milk in the reservoir and analyze it for quantity or other characteristics. The pump control 465 may be any of the pump controls described elsewhere in this applications, and the data may be processed, transmitted, or displayed using any of the methods disclosed herein.

In some exemplary embodiments, the pumping devices described herein can employ one or more capacitive sensors for measuring fluid volume. The capacitive sensors can be configured to detect the volume of fluid contained in any suitable portion of the pumping device, such as fluid contained within a collection reservoir and/or within a breast interface (e.g., expression area 260, a component permitting passage of fluid from the interface such as a valve, exit port, or tube).

FIGS. 11H-11I illustrate exemplary embodiments of expression devices that use capacitive sensors. The expression device 472 may be any of the expression devices disclosed herein and they have an interface 474 that also may be any of the interfaces disclosed in this specification. A reservoir 462 is threadably 466 or otherwise coupled to the expression device and the reservoir may be any of the reservoirs described herein. As milk 468 is expressed and collected at the outlet of the expression device, it passes through the capacitive sensor 475 which is then able to measure fluid volume. FIG. 11I is similar to the embodiment in FIG. 11H, with the major difference being that the capacitive sensor 475a is disposed in the reservoir 462 near the bottom, rather than in the outlet of the expression device. The data from the sensor in either embodiment may then be processed, transmitted, or displaying using any of the techniques described herein.

In other exemplary embodiments, one or more strain gauges can be used to measure the volume of expressed fluid. The strain gauges can be situated at any suitable position in the pumping device. For example, a strain gauge can be coupled to a flap valve (or any other valve permitting passage of expressed fluid) and configured to determine the volume based on the displacement of the valve over time. Alternatively or in addition, a strain gauge can be coupled to a collection reservoir and configured to measure the volume of expressed fluid contained within the reservoir.

FIG. 11J illustrates an exemplary embodiment of a strain gauge. The expression device 472 includes an interface 474 and reservoir 462 threadably 466 or otherwise coupled thereto. Any portion of this system may be any of the components described elsewhere in this specification. As milk is expressed 468 it accumulates in the outlet of the expression device. Eventually, the weight of the accumulated milk is sufficient to actuate and open valve 476. A strain gauge 481 is coupled to the flap valve and this sensor is then used to collect data on movement of the valve and therefore this correlates to the collected fluid. The fluid accumulates in a layer 464 in reservoir 462. The data from the sensor is then processed, transmitted, or displayed using any of the methods disclosed herein.

FIG. 11K illustrates an alternative embodiment of a strain gauge. This embodiment generally takes the same form as the previous embodiment with the major difference being that the collected fluid layer 464 is disposed over a plate 483 which bears the weight of the collected fluid. Thus, as the weight increases or decreases, a strain gauge 481a disposed under the plate 483 detects the weight change and this can be correlated to the collected fluid volume. Data from the sensor is then processed, transmitted, or displayed according to any of the methods disclosed herein.

FIGS. 11O-11Q illustrate an exemplary embodiment of a strain gauge or force sensitive resistor (FSR) comprising an integrated processing unit. FIG. 11O is a cross-sectional view the embodiment. The strain gauge 490 may be integrated into the bottom of a reservoir, such as reservoir 462 or any reservoir described herein, in such a configuration as to place the load 469 of the expressed milk 468 onto the sensor area of the strain gauge or FSR 490. The strain gauge 490 may comprise a small, force sensitive resistor that adjusts its resistance based on the compressive force it is under. In order to maximize the sensitivity of the strain gauge 490 to the load 469, the bottom interior surface 491 of the reservoir 462 may be designed in such a way as to minimize the absorption of the load 469 as the load is transmitted to the strain gauge. For example, the bottom interior surface 491 may comprise a bellows element 492 to allow the surface 491 to move up and down, thereby minimizing absorption of the load 469 by stretching the surface 491. Preferably, the reservoir 462 comprises self-contained electronics to collect, process, and communicate the data generated using the strain gauge 490. As shown in FIG. 11P, which is an exploded view of the embodiment in FIG. 11O, the strain gauge 490 may be mounted on a support 493, and coupled to a processing unit 494. Power may be supplied to the processing unit 494 via battery or a direct contact connection such as a cable or pad connectors, or, preferably, via an inductive charging system. The inductive charging system may comprise a battery 495 coupled to the processing unit, and a wireless charger 496 coupled to the battery, which may be charged using an inductive charging method as known in the art. FIG. 11Q is a detailed view of the processing unit 494. The processing unit 494 may comprise a printed circuit board (PCB) housing one or more of a microcontroller 494a, a communication module 494b, a strain gauge connection 494c, a power connection 494d, and a timer 494e. The processing unit 494 may receive signals from the strain gauge 490 through the strain gauge connection 494c, and the signals may be transmitted to the microcontroller 494a. The microcontroller 494a may comprise a non-transitory computer readable medium comprising instructions to collect and process the signals received from the strain gauge 490. The microcontroller 494a may further comprise instructions to transmit the collected and/or processed signals to the communication module 494b. The communication module 494b may comprise wireless transmitter/receiver such as a Blue Tooth module, for example. The communication module 494b may be configured to transmit the strain gauge data to a pump control unit of the expression device or to another computing device, such as a mobile phone, for data analysis and/or display.

The integrated processing unit of the embodiment of FIGS. 11O-11Q may also be suitably combined with any other sensor described herein. An expression device with a reservoir having an integrated sensor and processing unit as described can help automate the management and monitoring of milk production, thus reducing the need for manually maintaining records related to milk production. For example, the strain gauge system as described can monitor the quantity of milk produced, and automatically process and send the data to a computing device, from which the user may easily access the information. Such a system can greatly improve convenience for the users, and also help reduce human errors related to manual record maintenance.

In exemplary embodiments, some or all of the measurement data collected by the sensors can be fed back to the pumping device in order to optimize fluid expression. Preferably, the feedback can be transmitted to a processing unit and/or control unit of the pumping device (e.g., suitable hardware located in the controller 115) configured to control one or more functionalities of the actuation assembly. Based on the feedback, the processing unit can determine changes to actuation parameters of the actuation assembly in order to achieve and/or maintain optimal fluid expression. For example, the feedback can be used to determine adjustments to a vacuum stroke or the cycles per minute of a pump, piston assembly, or any other suitable actuation assembly.

FIG. 21 illustrates an exemplary expression system with feedback control. The system includes a pump unit 2100 preferably including a controller and processor 2104 as well as a motor 2102 for actuating the device, and a distal assembly 2110 which is sized and shaped to mate with the target anatomy, here a breast 2112. Any of the elements in this exemplary system may be any of the components disclosed elsewhere in this specification in other exemplary embodiments. In this embodiment, feedback 2106 from the sensor which monitors expressed milk in the expression device 2110 is transmitted from the distal assembly (expression device with interface) to the controller and processor 2104. The data is processed and this information is used to provide instructions to motor 2102 which increases or decreases actuation of the expression device which is then transmitted by communication 2108 back to the expression device or distal assembly 2110. The feedback information may also be used to provide instructions to motor 2012 to change the stroke or number of cycles per minute of the expression device. Any of the embodiments in this specification may include such a feedback loop.

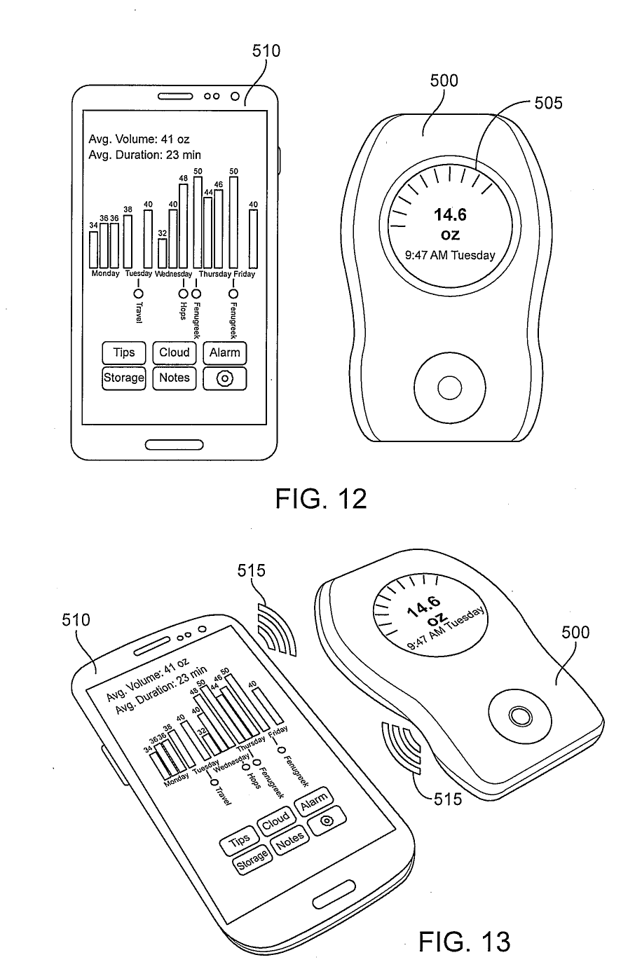

FIG. 12 illustrates an exemplary embodiment of a controller 500 for a pumping device including a display screen 505. The controller 500 can include suitable hardware for collecting, processing, and storing the milk expression data described herein, as well as analysis results obtained from processing the expression data. In preferred embodiments, this information is displayed to a user of the pumping device via the display screen 505. Furthermore, as shown in FIG. 13, information can also be transmitted from the controller 500 and displayed on a separate computing device, such as a mobile device 510, as described in further detail below. The information can be presented in any suitable format, including graphs, charts, tables, images, or other visual elements, such as one or more lights of different colors. Alternatively or in combination, the information may be provided via an audible indicator. The information may be presented in a format that is static or dynamic (e.g., updated in real time, etc.). Additionally, the controller 500 can include input devices enabling users to interact with the displayed information, such as the button 515, as well as keyboards, joysticks, touchscreens, switches, or knobs, or suitable combinations thereof.

Communication with Computing Devices

In any of the embodiments disclosed herein, the pumping devices described herein can be configured to communicate with another entity, such as one or more computing devices and/or servers. Exemplary computing devices include personal computers, laptops, tablets, and mobile devices (e.g., smartphones, cellular phones). The servers described herein can be implemented across physical hardware, virtualized computing resources (e.g., virtual machines), or any suitable combination thereof. In preferred embodiments, the servers are distributed computing servers (also known as cloud servers) utilizing any suitable combination of public and/or private distributed computing resources. The computing devices and/or servers may be in close proximity to the pumping device (short range communication), or may be situated remotely from the pumping device (long range communication). Any description herein relating to communication between a computing device and a pumping device can also be applied to communication between a server and a pumping device, and vice-versa.

FIG. 13 illustrates short range communication 515 between the controller 500 of a pumping device and mobile device 510. The communication 515 can utilize wireless communication methods, as described below. In many embodiments, the controller 500 and mobile device 510 are also capable of long range communication.

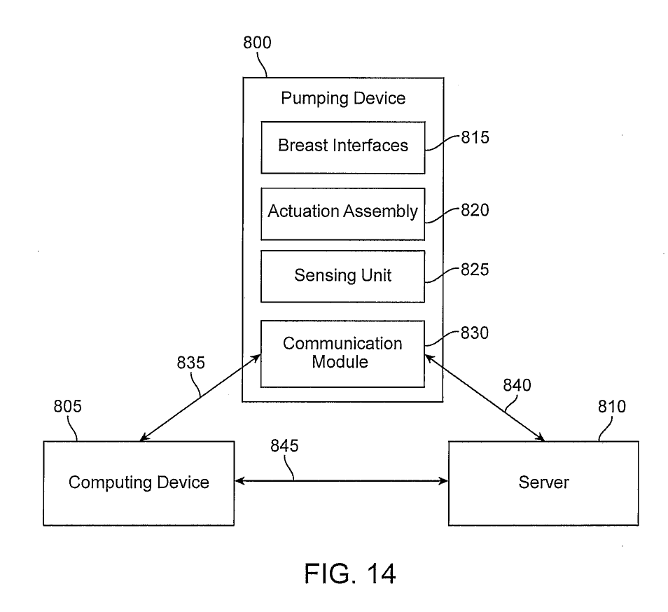

FIG. 14 is a schematic illustration of a pumping device 800 in communication with a computing device 805 and a server 810. The pumping device 800 includes one or more breast interfaces 815, an actuation assembly 820, a sensing unit 825, and a communication module 835. Preferably, the communication module 830 is implemented across suitable hardware within a controller of the pumping device (e.g., controller 500). The pumping device 800 can communicate with the computing device 805 and server 810 via the communication module 830. In many embodiments, the communication module 830 is communicably coupled to the computing device 805 and server 810 via first and second data connections 835, 840. Furthermore, the server 810 can be communicably coupled to the computing device 805 via a third data connection 845. Although the pumping device 800 is depicted herein as communicating directly with the computing device 805 and the server 810, other configurations are also possible. For example, the pumping device 800 may communicate with the server 810 indirectly via the computing device 805, or vice-versa. Conversely, the server 810 may communicate with the pumping device 800 indirectly via the computing device 805, and the computing device 805 may communicate with the pumping device 800 via the server 810. Any description herein related to communication between the pumping device 800, the computing device 805, or server 810 can be applied to direct communication as well as indirect communication between these entities.

The data connections 835, 840, and 845 can utilize any communication method suitable for transmitting data between the pumping device 800, the computing device 805, and server 810. Such communication methods can include wired communication (e.g., wires, cables such as USB cables, fiber optics) and/or wireless communication (Bluetooth®, WiFi, near field communication). In many embodiments, data can be transmitted over one or more networks, such as local area networks (LANs), wide area networks (WANs), telecommunications networks, the Internet, or suitable combinations thereof.

In exemplary embodiments, the pumping device 800 transmits milk expression data to the computing device 805 or server 810 (directly or indirectly). The milk expression data can include measurement data generated by the sensing unit 825 of the pumping device 800, as previously described herein. In many embodiments, the pumping device 800 analyzes the measurement data (e.g., using suitable onboard hardware and/or software) and transmits the analysis results to the computing device 805 or server 810. Alternatively, the measurement data can be analyzed by the computing device 805 or server 810, such as using one or more applications. The computing device 805 or server 810 may be associated with data stores for storage of the measurement data and/or analysis results.

The applications (of the computing device 805 or server 810) can also collect and aggregate the measurement data and/or analysis results and display them in a suitable format to a user (e.g., charts, tables, graphs, images, etc.), as previously described herein. Preferably, the application includes additional features that allow the user to overlay information such as lifestyle choices, diet, and strategies for increasing milk production, in order to facilitate the comparison of such information with milk production statistics. The analysis and display functionalities described herein may be performed by a single entity, or by any suitable combination of entities. For example, in many embodiments, data analysis can be carried out by the server 810, and the analysis results transmitted to the pumping device 800 or computing device 805 for display to the user.

Additionally, the computing device 805 or server 810 can include an application configured to control at least one functionality of the pumping device 800 or a portion thereof (e.g., the actuation assembly 820), such as power, vacuum pressure applied (via the interfaces 815), or cycles per minute. For example, the communication module 830 can receive control signals from the computing device 805 and/or sever 810, and transmit the control signals to the actuation assembly 820 to produce the desired actuation. In preferred embodiments, the control signals can be generated using feedback provided by the pumping device 800, such as feedback based on measurement data provided by the sensing unit 825, as previously described herein. Additionally, the computing device 805 or server 810 may implement machine learning techniques with regards to control of the pumping device 800, in order to improve and optimize pumping performance over time.

Furthermore, the pumping device 800, computing device 805, and/or server 810 can be configured to provide notifications reminding the user to express milk. Such notifications can help avoid missed pumping sessions, and thus reduce the incidence of associated complications such as mastitis. The notifications can be generated based on previously collected milk expression data, such as data relating to expression frequency and/or the timing of previous pumping sessions, as well as based on user preferences. Preferably, the notification functionality is included in a suitable application running on the computing device 805 or server 810. For example, the pumping device 800 can send information about the times of pump usage to the computing device 805 or server 810, so that the application can identify when pumping has occurred and set reminders at desired pumping times.

The notifications can be provided using any suitable method and in any suitable format. For example, the notifications can be generated by the computing device 805 or server 810, transmitted to the pumping device 800 (e.g., to the communication module 830), and displayed to the user (e.g., on a display of the pumping device 800, such as the display screen 505). Conversely, the notifications can be generated by the pumping device 800 and transmitted to the computing device 805 and/or server 810. In many embodiments, the notifications are displayed to the user by the computing device 805. Alternatively, the pumping device 800, computing device 805 and/or sever 810 can provide the notifications to the user using other methods. For example, the notifications can be sent to an email address, via short message service (SMS) to a smartphone or other mobile device associated with a cellular phone number, or to a web page accessible by the user.

Other types of data can also be transmitted between the pumping device 800, computing device 805, and/or server 810. For example, in many embodiments, firmware updates for one or more components of the pumping device 800 can be transmitted to the pumping device 800 from the computing device 805 and/or server 810.

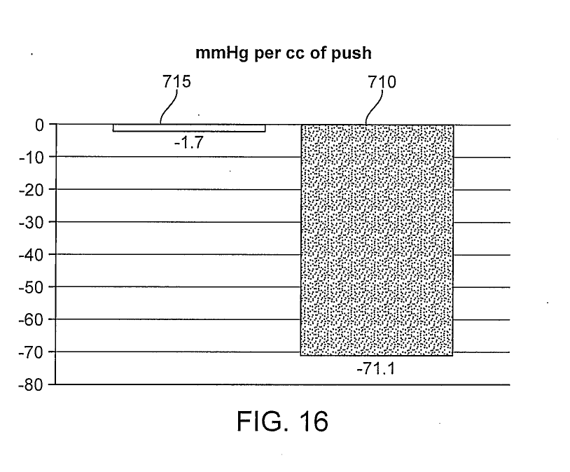

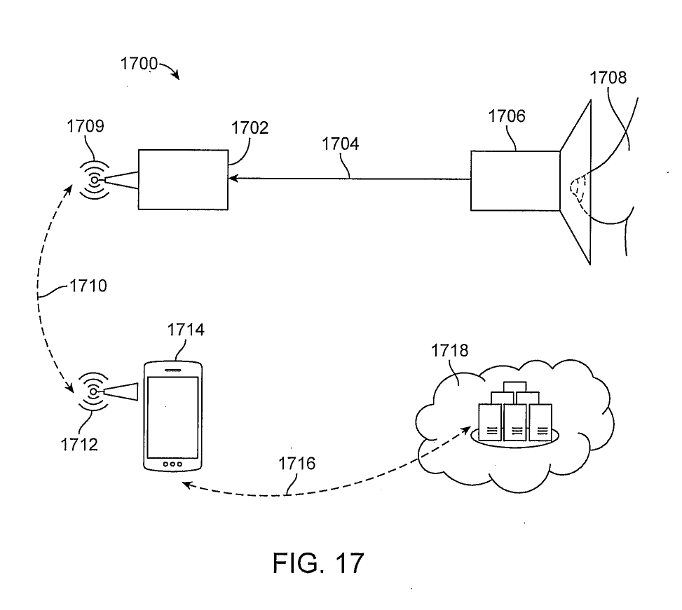

FIG. 17 illustrates another exemplary embodiment of a system for expression of milk or for monitoring other fluids. The system 1700 includes a pump unit 1702, a distal assembly 1706 (sometimes also referred to as an interface in this specification), wireless communication transmitters and receivers 1709, 1712, a computing device 1714 and a remote server 1718. The pump unit 1702 may be any of the pump units described in this specification or known in the art, and the distal assembly 1708 also may be any of those described herein or known in the art. The distal assembly 1706 is preferably sized and shaped to conform to the target anatomy, which in this exemplary embodiment is a breast 1708. The pump unit 1702 actuates 1704 the distal assembly 1706 to cause expression of milk from breast 1708 using any of the actuation mechanism disclosed herein. A transmitter 1709 is preferably disposed on the pump unit or adjacent thereto and is configured to transmit data 1710 from the pump unit to a receiver 1712 on the computing device 1714. The data may be transmitted wirelessly using methods known in the art such as those disclosed in this specification. In alternative embodiments, a hard connection such as with a USB cable may be used to operably couple the pump 1702 and computing device 1714 together. The computing device may be a smart phone, tablet, personal computer, or any other electronic computing device that can display the data transmitted from the pump unit 1702. The computing device may also transmit information back to the pump unit to help control operation of the distal assembly. The computing device 1714 may also communicate 1716 with a remote server 1718 which may store or display the data. Access to the remote service 1718 may be by the Internet or by other means known in the art and thus the cloud based data may be readily accessed from any other device with Internet access.

FIG. 18 illustrates another exemplary embodiment of a system 1800 for expression of milk. In this embodiment, the system 1800 includes a pump unit 1802, a distal assembly 1806 and a cloud based or remote server 1812. The pump unit 1802 may be any of the pumps disclosed herein and it is operably coupled with the distal assembly 1806 which is sized and shaped to conform to the target, such as breast 1808. The distal assembly may be any of the distal assemblies described herein. The pump unit 1802 actuates 1804 the distal assembly using any of the mechanisms disclosed herein to cause expression of milk from breast 1808. The pump unit 1802 also includes a transmitter and receiver 1809 for transmitting pump data 1810 to a remove server 1812, which in this embodiment is a cloud based server. Thus, the data may be transmitted to the remote service via the Internet, and accessed from the cloud based server by the pump 1802 or any other computing device via the Internet. Preferably communication with the cloud based server is by wireless communication.

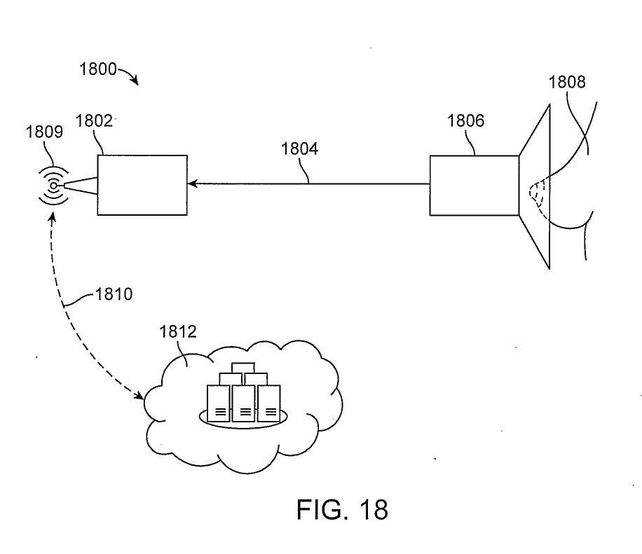

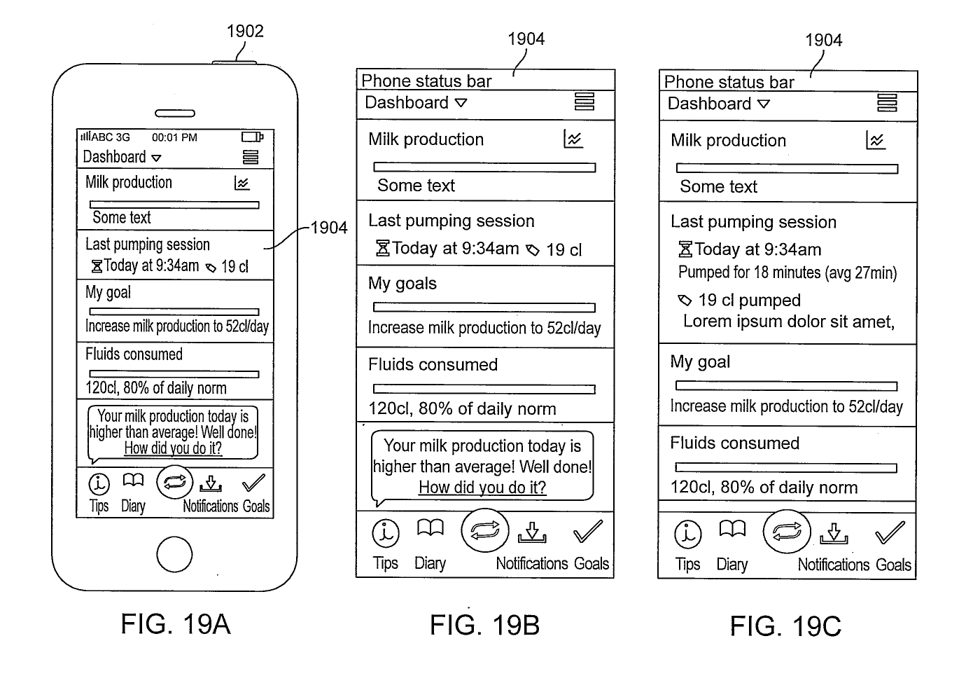

FIGS. 19A-19C illustrate exemplary computing device displays 1904. For example, FIG. 19A illustrates an exemplary display on a mobile phone 1902 and graphically illustrates milk production, the time of the last pumping session, a graphic of goal attainment, and a graphic illustrating the fluid consumption of the user. Additionally, the display 1904 may also provide user encouragement or user feedback based on the amount of milk production. FIG. 19B is an enlarged view of the display 1904 in FIG. 19A. FIG. 19C illustrates additional information that the display 1904 may show when a touch screen is actuated (e.g. by swiping or touching the screen). For example, the volume of the milk expressed is indicated after the “Last Pumping Session” section of the display is selected. Some or all items may be expanded, as also indicated in FIG. 19C. Additional information, or in some situations, less information may be displayed as desired.

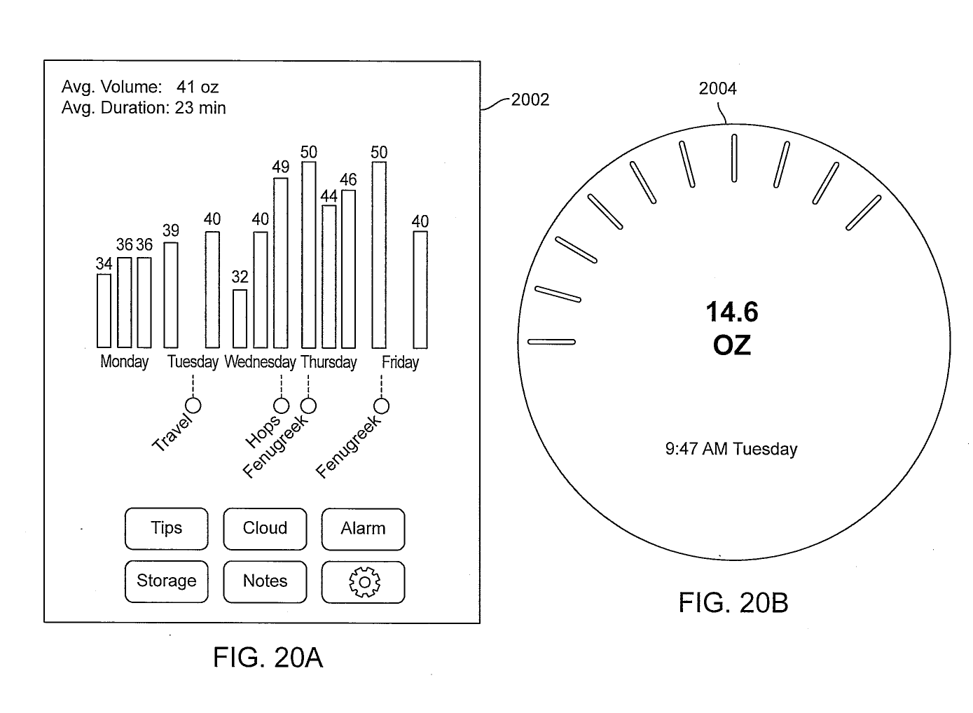

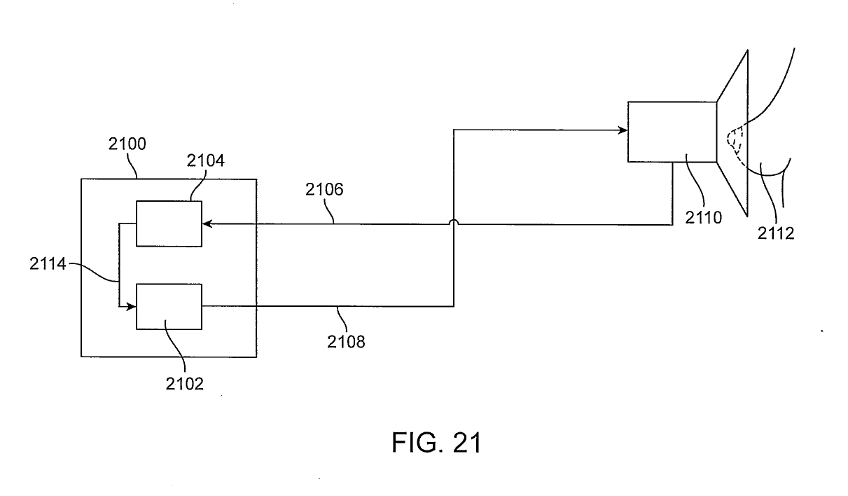

FIGS. 20A-20B illustrate other exemplary displays which may be used in a milk expression system. For example, FIG. 20A is an exemplary display 2002 on any of the computing devices disclosed herein and operably coupled with any of the pump units described herein. The display may indicate an average volume of milk expressed over any time period, along with an average duration of the expression session during that same time period. Graphics may be used (e.g. bar chart, pie chart, x-y plot, etc.) to show volume expressed during individual sessions over the course of several days, here Monday through Friday. The display may allow a user to annotate the display so that missed sessions may be accounted for, for example if a session is omitted due to traveling, the display may show travel during that time period. Other annotations may also be made, such as when certain foods or nutritional supplements are taken, here hops or fenugreek. This allows the user to recall when expressed milk samples were obtained relative to the consumption of the food or nutritional supplements. The display may have other functional buttons such as for obtaining tips, accessing the cloud, setting an alarm, making notes, storing data, or establishing system preferences. Communication between the computing device and the pump unit in FIGS. 20A-20B is discussed more thoroughly above in relation to FIG. 13.

FIG. 20B illustrates an exemplary display 2004 that may be on a computing device in the system, or more preferably that is on any of the pumps disclosed herein. The display 2004 is similar to a dashboard style gauge and indicates the volume of fluid expressed and collected and the time. Other information may also be displayed.

Experimental Data

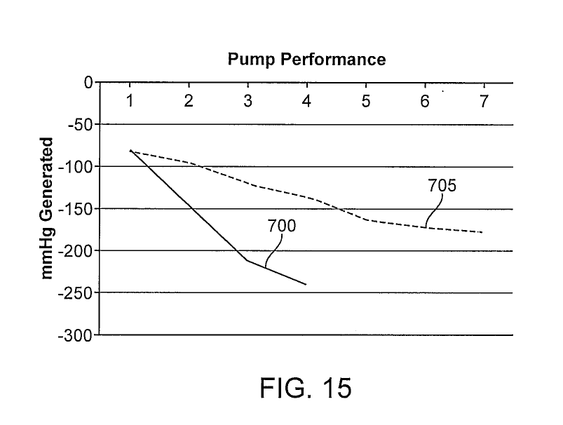

FIGS. 15 and 16 illustrate experimental pumping data obtained from a commercial breast pump device and an exemplary embodiment of the present invention. The exemplary embodiment utilized an incompressible fluid for pumping and had a maximum hydraulic fluid volume of 4 cc, while the commercial device utilized air for pumping and had a maximum volume of 114 cc.

FIG. 15 illustrates a graph of the pump performance as quantified by vacuum pressure generated per run. For the exemplary embodiment, pressure measurements were taken for 1 cc, 2 cc, 3 cc, and 4 cc of fluid volume displaced by the pump, with the run number corresponding to the volume in cc. For the commercial device, measurements were taken with the pump set to one of seven equally incremented positions along the vacuum adjustment gauge representing 46 cc, 57 cc, 68 cc, 80 cc, 91 cc, 103 cc, and 114 cc of fluid volume displaced by the pump, respectively, with the run number corresponding to the position number. Curve 700 corresponds to the exemplary embodiment and curve 705 corresponds to the commercial device. The exemplary embodiment generated higher levels of vacuum pressure per displacement volume compared to the commercial device, with maximum vacuum pressures of −240.5 mmHg and −177.9 mmHg, respectively.

FIG. 16 illustrates a graph of the pump efficiency as measured by the maximum vacuum pressure per maximum volume of fluid displaced, with bar 710 corresponding to the exemplary embodiment and bar 715 corresponding to the commercial device. The exemplary embodiment demonstrated a 42-fold increase in pumping efficiency compared to the commercial device, with efficiencies of −71.1 mmHg/cc and −1.7 mmHg/cc, respectively.

The various techniques described herein may be partially or fully implemented using code that is storable upon storage media and computer readable media, and executable by one or more processors of a computer system. Storage media and computer readable media for containing code, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices, solid state drives (SSD) or other solid state storage devices, or any other medium which can be used to store the desired information and which can be accessed by the a system device. Based on the disclosure and teachings provided herein, a person of ordinary skill in the art will appreciate other ways and/or methods to implement the various embodiments.

It shall be understood that different aspects of the invention can be appreciated individually, collectively, or in combination with each other. Suitable elements or features of any of the embodiments described herein can be combined or substituted with elements or features of any other embodiment.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A system for expression of milk from a breast, the system comprising:
an expression apparatus comprising an interface, an actuation assembly operably coupled to the interface, and a sensing unit, wherein actuation of the actuation assembly causes the interface to apply vacuum pressure at the breast to express milk therefrom; and
a computing device configured to communicate with the expression apparatus via a data connection;
wherein the sensing unit is configured to generate measurement data indicative of one or more characteristics of milk expression or of the expressed milk, and
wherein the expression apparatus comprises a communication module configured to transmit the measurement data to the computing device via the data connection.

2. (canceled)

3. The system of claim 1, wherein the data connection utilizes one or more of a wireless communication, near field communication, and a USB cable to transmit data between at least a portion of the expression apparatus and the computing device.

4. The system of claim 1, wherein the computing device is selected from a smartphone, a tablet, and a personal computer.

5.-6. (canceled)

7. The system of claim 16, wherein the computing device comprises an application configured to analyze the measurement data and thereby generate an analysis result.

8. The system of claim 7, further comprising a server in communication with the computing device via a network, wherein the computing device is configured to transmit the analysis result to the server via the network.

9.-11. (canceled)

12. The system of claim 7, wherein the computing device is configured to store the analysis result.

13. The system of claim 7, wherein the computing device is configured to display the analysis result to a user.

14.-21. (canceled)

22. A method for expression of milk from a breast, the method comprising:
providing a expression apparatus comprising an interface, an actuation assembly operably coupled to the interface, and a sensing unit;
engaging the interface with a breast;
actuating the actuation assembly, thereby causing the interface to apply vacuum pressure at the breast;
expressing milk from the breast; and
measuring, using the sensing unit, one or more characteristic of milk expression or of the expressed milk, thereby generating measurement data.

23. The method of claim 22, further comprising transmitting the measurement data to a computing device in communication with a communication module of the expression apparatus via a data connection.

24. The method of claim 22, further comprising analyzing the measurement data via an application of the computing device to generate an analysis result.

25. The method of claim 24, further comprising displaying the analysis result to a user via the computing device.

26. The method of claim 25, wherein the analysis result is displayed in a graph, chart, or table.

27. The method of claim 24, further comprising storing the analysis result in one or more data stores of the computing device.

28. The method of claim 24, further comprising transmitting the analysis result to a server in communication with the computing device via a network.

29. The method of claim 22, further comprising storing the measurement data in a processing unit of the expression device.

30. The method of claim 22, further comprising storing the measurement data in one or more data stores of the computing device.

31.-46. (canceled)

47. The method of claim 22, wherein the sensing unit is coupled to a reservoir in fluid communication with the interface, and wherein the one or more characteristic of milk expression or of the expressed milk are measured by the sensing unit as the expressed milk moves from the interface to the reservoir.

48. The method of claim 47, further comprising storing the measurement data on a processing unit of the reservoir.

49. The method of claim 17, further comprising transmitting the measurement data to a computing device in communication with a communication module of the reservoir via a data connection.

50.-51. (canceled)

52. An apparatus for expression of milk from a breast, the apparatus comprising:
an interface configured to engage a breast;
an actuation assembly operably coupled to the interface, wherein actuation of the actuation assembly causes the interface to apply vacuum pressure at the breast to express fluid therefrom; and
a sensing unit configured to generate measurement data indicative of one or more characteristics of milk expression or of the expressed milk.

53. The apparatus of claim 52, wherein the one or more characteristics of the expressed milk include one or more of a volume or a weight of the expressed milk, and wherein the one or more characteristics of milk expression include one or more of expression frequency, expression date, expression time, expression duration, or a position or motion of a portion of the expression apparatus during milk expression.

54. (canceled)

55. The apparatus of claim 52, wherein the measurement data is indicative of a volume of the expressed milk.

56. The apparatus of claim 55, wherein the measurement data is indicative of the volume per unit time of the expressed milk.

57.-68. (canceled)

69. The apparatus of claim 52, further comprising a reservoir in fluid communication with the interface and configured to collect the expressed milk, wherein the sensing unit is coupled to the reservoir, and wherein the reservoir comprises a processing unit in communication with the sensing unit, the processing unit configured to receive the measurement data generated by the sensing unit.

70. The apparatus of claim 69, wherein the processing unit comprises a communication module configured to transmit the measurement data to a computing device via a data connection.

71. The apparatus of claim 69, wherein the processing unit comprises a communication module configured to transmit the measurement data to a server via a network.

72. The apparatus of claim 69, wherein the processing unit is configured to store the measurement data.

73.-76. (canceled)

77. The apparatus of claim 69, wherein the sensing unit comprises a capacitive sensor configured to measure a volume of the expressed milk contained in the reservoir.

78.-86. (canceled)

87. The apparatus of claim 52, further comprising:
a processing unit; and
a control unit operably coupled to the actuation assembly to control at least one functionality thereof;
wherein at least a subset of the measurement data is transmitted as feedback to at least one of the processing unit and the control unit.

88. The apparatus of claim 87, wherein the actuation assembly comprises a pump, and the feedback is used to adjust a vacuum stroke of the pump to maintain optimal fluid expression.

89. The apparatus of claim 87, wherein the actuation assembly comprises a pump, and the feedback is used to adjust cycles per minute of the pump to maintain optimal fluid expression.

90.-91. (canceled)
